(19) United States
(12) Patent Application Publication
(10) Pub. No.: US 2024/0153577 A1
(43) Pub. Date: May 9, 2024

(54) PREDICTING SYMMETRICAL PROTEIN STRUCTURES USING SYMMETRICAL EXPANSION TRANSFORMATIONS

(71) Applicant: DeepMind Technologies Limited, London (GB)

(72) Inventors: Richard Andrew Evans, London (GB); Alexander Pritzel, London (GB); Russell James Bates, London (GB); John Jumper, London (GB)

(21) Appl. No.: 18/027,571

(22) PCT Filed: Nov. 23, 2021
(86) PCT No.: PCT/EP2021/082618

§ 371 Date: Mar. 21, 2023

Related U.S. Application Data
(60) Provisional application No. 63/118,914, filed on Nov. 28, 2020.

(51) Int. Cl. G16B 15/20; G16B 40/20

(52) U.S. Cl. CPC G16B 15/20; G16B 40/20

(57) ABSTRACT

Methods, systems, and apparatus, including computer programs encoded on a computer storage medium, for predicting a structure of a protein that comprises a plurality of amino acid chains. According to one aspect, a method comprises: obtaining initial structure parameters for a first amino acid chain in the protein; obtaining data identifying a symmetry group; processing the initial structure parameters for the first amino acid chain and the data identifying the symmetry group using a folding neural network that comprises a sequence of up-date blocks, wherein each update block performs operations comprising: applying a symmetrical expansion transformation to the current structure parameters for the first amino acid chain; and processing the current structure parameters for the amino acid chains in the protein, in accordance with values of the update block parameters of the update block, to update the current structure parameters for the

### CROSS-REFERENCE TO RELATED APPLICATIONS

This application claims the benefit of the filing date of U.S. Provisional Patent Application Ser. No. 63/118,914, which was filed on Nov. 28, 2020, and which is incorporated herein by reference in its entirety.

### BACKGROUND

This specification relates to predicting protein structures.

A protein is specified by one or more sequences (“chains”) of amino acids. An amino acid is an organic compound which includes an amino functional group and a carboxyl functional group, as well as a side chain (i.e., group of atoms) that is specific to the amino acid. Protein folding refers to a physical process by which one or more sequences of amino acids fold into a three-dimensional (3-D) configuration. The structure of a protein defines the 3-D configuration of the atoms in the amino acid sequences of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a protein structure prediction system implemented as computer programs on one or more computers in one or more locations that can predict the structures of symmetrical proteins.

The term “protein” can be understood to refer to any biological molecule that is specified by one or more sequences (or “chains”) of amino acids. For example, the term protein can refer to a protein domain, e.g., a portion of an amino acid chain of a protein that can undergo protein folding nearly independently of the rest of the protein. As another example, the term protein can refer to a protein complex, i.e., that includes multiple amino acid chains that jointly fold into a protein structure.

A “multiple sequence alignment” (MSA) for an amino acid sequence in a protein specifies a sequence alignment of the amino acid sequence with multiple additional amino acid sequences, referred to herein as “MSA sequences,” e.g., from other proteins, e.g., homologous proteins. More specifically, the MSA can define a correspondence between the positions in the amino acid chain and corresponding positions in multiple MSA sequences. A MSA for an amino acid sequence can be generated, e.g., by processing a database of amino acid sequences using any appropriate computational sequence alignment technique, e.g., progressive alignment construction. The MSA sequences can be understood as having an evolutionary relationship, e.g., where each MSA sequence may share a common ancestor. The correlations between the amino acids in the MSA sequences for an amino acid chain can encode information that is relevant to predicting the structure of the amino acid chain.

An “embedding” of an entity (e.g., a pair of amino acids) can refer to a representation of the entity as an ordered collection of numerical values, e.g., a vector or matrix of numerical values.

The structure of a protein can be defined by a set of structure parameters. A set of structure parameters defining the structure of a protein can be represented as an ordered collection of numerical values. A few examples of possible structure parameters for defining the structure of a protein are described in more detail next.

In one example, the structure parameters defining the structure of a protein include: (i) location parameters, and (ii) rotation parameters, for each amino acid in the protein.

The location parameters for an amino acid can specify a predicted 3-D spatial location of a specified atom in the amino acid in the structure of the protein. The specified atom can be the alpha carbon atom in the amino acid, i.e., the carbon atom in the amino acid to which the amino functional group, the carboxyl functional group, and the side chain are bonded. The location parameters for an amino acid can be represented in any appropriate coordinate system, e.g., a three-dimensional [x, y, z] Cartesian coordinate system.

The rotation parameters for an amino acid can specify the predicted “orientation” of the amino acid in the structure of the protein. More specifically, the rotation parameters can specify a 3-D spatial rotation operation that, if applied to the coordinate system of the location parameters, causes the three “main chain” atoms in the amino acid to assume fixed positions relative to the rotated coordinate system. The three main chain atoms in the amino acid can refer to the linked series of nitrogen, alpha carbon, and carbonyl carbon atoms in the amino acid. The rotation parameters for an amino acid can be represented, e.g., as an orthonormal 3×3 matrix with determinant equal to 1.

Generally, the location and rotation parameters for an amino acid define an egocentric reference frame for the amino acid. In this reference frame, the side chain for each amino acid may start at the origin, and the first bond along the side chain (i.e., the alpha carbon—beta carbon bond) may be along a defined direction.

In another example, the structure parameters defining the structure of a protein can include a “distance map” that characterizes a respective estimated distance (e.g., measured in angstroms) between each pair of amino acids in the protein. A distance map can characterize the estimated distance between a pair of amino acids, e.g., by a probability distribution over a set of possible distances between the pair of amino acids.

In another example, the structure parameters defining the structure of a protein can define a three-dimensional (3D) spatial location of each atom in each amino acid in the structure of the protein.

The protein structure prediction system described herein can be used to obtain a ligand such as a drug or a ligand of an industrial enzyme. For example, a method of obtaining a ligand may include obtaining a target amino acid sequence, in particular the amino acid sequence of a target protein, e.g. a drug target, and processing an input based on the target amino acid sequence using the protein structure prediction system to determine a (tertiary) structure of the target protein, i.e., the predicted protein structure. The method may then include evaluating an interaction of one or more candidate ligands with the structure of the target protein. The method may further include selecting one or more of the candidate ligands as the ligand dependent on a result of the evaluating of the interaction.

In some implementations, evaluating the interaction may include evaluating binding of the candidate ligand with the structure of the target protein. For example, evaluating the interaction may include identifying a ligand that binds with sufficient affinity for a biological effect. In some other implementations, evaluating the interaction may include evaluating an association of the candidate ligand with the structure of the target protein which has an effect on a function of the target protein, e.g., an enzyme. The evaluating may include evaluating an affinity between the candidate ligand and the structure of the target protein, or evaluating a selectivity of the interaction. The candidate ligand(s) may be selected according to which have the highest affinity.

The candidate ligand(s) may be derived from a database of candidate ligands, and/or may be derived by modifying ligands in a database of candidate ligands, e.g., by modifying a structure or amino acid sequence of a candidate ligand, and/or may be derived by stepwise or iterative assembly/optimization of a candidate ligand.

The evaluation of the interaction of a candidate ligand with the structure of the target protein may be performed using a computer-aided approach in which graphical models of the candidate ligand and target protein structure are displayed for user-manipulation, and/or the evaluation may be performed partially or completely automatically, for example using standard molecular (protein-ligand) docking software. In some implementations the evaluation may include determining an interaction score for the candidate ligand, where the interaction score includes a measure of an interaction between the candidate ligand and the target protein. The interaction score may be dependent upon a strength and/or specificity of the interaction, e.g., a score dependent on binding free energy. A candidate ligand may be selected dependent upon its score.

In some implementations the target protein includes a receptor or enzyme and the ligand is an agonist or antagonist of the receptor or enzyme. In some implementations the method may be used to identify the structure of a cell surface marker. This may then be used to identify a ligand, e.g., an antibody or a label such as a fluorescent label, which binds to the cell surface marker. This may be used to identify and/or treat cancerous cells.

In some implementations the ligand is a drug and the predicted structure of each of a plurality of target proteins is determined, and the interaction of the one or more candidate ligands with the predicted structure of each of the target proteins is evaluated. Then one or more of the candidate ligands may be selected either to obtain a ligand that (functionally) interacts with each of the target proteins, or to obtain a ligand that (functionally) interacts with only one of the target proteins. For example in some implementations it may be desirable to obtain a drug that is effective against multiple drug targets. Also or instead it may be desirable to screen a drug for off-target effects. For example in agriculture it can be useful to determine that a drug designed for use with one plant species does not interact with another, different plant species and/or an animal species.

In some implementations the ligand is a drug and the predicted structure of a target protein that is a protein complex, e.g. a dimer or multimer, is determined. Evaluating the interaction of the one or more candidate ligands with the predicted structure of the target protein may then comprise identifying a candidate ligand that interacts with the protein complex, and that might therefore be expected to affect the formation or stability of the complex. This could afterwards be confirmed by experimental screening. Thus such a process may be used to identify a drug which is able to disrupt a protein complex or inhibit formation of the complex. Some diseases, e.g. neuro degenerative diseases such as dementia, are caused by protein aggregation. The method may thus be used to identify a ligand that is a drug to treat such a disease.

In some implementations the candidate ligand(s) may include small molecule ligands, e.g., organic compounds with a molecular weight of <900 daltons. In some other implementations the candidate ligand(s) may include polypeptide ligands, i.e., defined by an amino acid sequence.

In some cases, the protein structure prediction system can be used to determine the structure of a candidate polypeptide ligand, e.g., a drug or a ligand of an industrial enzyme. The interaction of this with a target protein structure may then be evaluated; the target protein structure may have been determined using a structure prediction neural network or using conventional physical investigation techniques such as x-ray crystallography and/or magnetic resonance techniques or cryogenic electron microscopy.

In another aspect there is provided a method of using a protein structure prediction system to obtain a polypeptide ligand (e.g., the molecule or its sequence). The method may include obtaining an amino acid sequence of one or more candidate polypeptide ligands. The method may further include using the protein structure prediction system to determine (tertiary) structures of the candidate polypeptide ligands. The method may further include obtaining a target protein structure of a target protein, in silico and/or by physical investigation, and evaluating an interaction between the structure of each of the one or more candidate polypeptide ligands and the target protein structure. The method may further include selecting one or more of the candidate polypeptide ligands as the polypeptide ligand dependent on a result of the evaluation.

As before evaluating the interaction may include evaluating binding of the candidate polypeptide ligand with the structure of the target protein, e.g., identifying a ligand that binds with sufficient affinity for a biological effect, and/or evaluating an association of the candidate polypeptide ligand with the structure of the target protein which has an effect on a function of the target protein, e.g., an enzyme, and/or evaluating an affinity between the candidate polypeptide ligand and the structure of the target protein, or evaluating a selectivity of the interaction. In some implementations the polypeptide ligand may be an aptamer. Again the polypeptide candidate ligand(s) may be selected according to which have the highest affinity.

As before the selected polypeptide ligand may comprise a receptor or enzyme and the ligand may be an agonist or antagonist of the receptor or enzyme. In some implementations the polypeptide ligand may comprises an antibody and the target protein comprises an antibody target, for example a virus, in particular a virus coat protein, or a protein expressed on a cancer cell. In these implementations the antibody binds to the antibody target to provide a therapeutic effect. For example, the antibody may bind to the target and act as an agonist for a particular receptor; alternatively, the antibody may prevent binding of another ligand to the target, and hence prevent activation of a relevant biological pathway.

Implementations of the method may further include synthesizing, i.e., making, the small molecule or polypeptide ligand. The ligand may be synthesized by any conventional chemical techniques and/or may already be available, e.g., may be from a compound library or may have been synthesized using combinatorial chemistry.

The method may further include testing the ligand for biological activity in vitro and/or in vivo. For example the ligand may be tested for ADME (absorption, distribution, metabolism, excretion) and/or toxicological properties, to screen out unsuitable ligands. The testing may include, e.g., bringing the candidate small molecule or polypeptide ligand into contact with the target protein and measuring a change in expression or activity of the protein.

In some implementations a candidate (polypeptide) ligand may include: an isolated antibody, a fragment of an isolated antibody, a single variable domain antibody, a bi- or multi-specific antibody, a multivalent antibody, a dual variable domain antibody, an immuno-conjugate, a fibronectin molecule, an adnectin, an DARPin, an avimer, an affibody, an anticalin, an affilin, a protein epitope mimetic or combinations thereof. A candidate (polypeptide) ligand may include an antibody with a mutated or chemically modified amino acid Fc region, e.g., which prevents or decreases ADCC (antibody-dependent cellular cytotoxicity) activity and/or increases half-life when compared with a wild type Fc region. Candidate (polypeptide) ligands may include antibodies with different CDRs (Complementarity-Determining Regions).

The protein structure prediction system described herein can also be used to obtain a diagnostic antibody marker of a disease. There is also provided a method that, for each of one or more candidate antibodies e.g. as described above, uses the protein structure prediction system to determine a predicted structure of the candidate antibody. The method may also involve obtaining a target protein structure of a target protein, evaluating an interaction between the predicted structure of each of the one or more candidate antibodies and the target protein structure, and selecting one of the one or more of the candidate antibodies as the diagnostic antibody marker dependent on a result of the evaluating, e.g. selecting one or more candidate antibodies that have the highest affinity to the target protein structure. The method may include making the diagnostic antibody marker. The diagnostic antibody marker may be used to diagnose a disease by detecting whether it binds to the target protein in a sample obtained from a patient, e.g. a sample of bodily fluid. As described above, a corresponding technique can be used to obtain a therapeutic antibody (polypeptide ligand).

Misfolded proteins are associated with a number of diseases. Thus in a further aspect there is provided a method of using the protein structure prediction system to identify the presence of a protein mis-folding disease. The method may include obtaining an amino acid sequence of a protein and using the protein structure prediction system to determine a structure of the protein. The method may further include obtaining a structure of a version of the protein obtained from a human or animal body, e.g., by conventional (physical) methods. The method may then include comparing the structure of the protein with the structure of the version obtained from the body and identifying the presence of a protein mis-folding disease dependent upon a result of the comparison. That is, mis-folding of the version of the protein from the body may be determined by comparison with the in silico determined structure.

In general identifying the presence of a protein mis-folding disease may involve obtaining an amino acid sequence of a protein, using an amino acid sequence of the protein to determine a structure of the protein, as described herein, and comparing the structure of the protein with the structure of a baseline version of the protein, identifying the presence of a protein mis-folding disease dependent upon a result of the comparison. For example the compared structures may be those of a mutant and wild-type protein. In implementations the wild-type protein may be used as the baseline version but in principle either may be used as the baseline version.

In some other aspects a computer-implemented method as described above or herein may be used to identify active/binding/blocking sites on a target protein from its amino acid sequence.

According to one aspect, there is provided a method performed by one or more data processing apparatus for predicting a structure of a protein that comprises a plurality of amino acid chains, the method comprising: obtaining initial structure parameters for a first amino acid chain in the protein, wherein the structure parameters for the first amino acid chain in the protein define predicted three-dimensional (3D) spatial locations of amino acids in the first amino acid chain in a structure of the protein; obtaining data identifying a symmetry group, wherein the protein is predicted to fold into a structure that is symmetrical with respect to the symmetry group; processing an input comprising the initial structure parameters for the first amino acid chain and the data identifying the symmetry group using a folding neural network to generate an output that defines a final predicted structure of the protein that is symmetrical with respect to the symmetry group, wherein the folding neural network comprises a sequence of update blocks, wherein each update block in the sequence of update blocks has a plurality of update block parameters and performs operations comprising: receiving current structure parameters for the first amino acid chain and the data identifying the symmetry group; applying a symmetrical expansion transformation to the current structure parameters for the first amino acid chain to generate respective current structure parameters for each other amino acid chain in the protein to define a current predicted structure of the protein that is symmetrical with respect to the symmetry group; and processing the current structure parameters for the amino acid chains in the protein, in accordance with values of the update block parameters of the update block, to update the current structure parameters for the first amino acid chain.

In some implementations, the structure parameters for the first amino acid chain in the protein include respective amino acid structure parameters for each amino acid in the first amino acid chain, and the amino acid structure parameters for each amino acid define a 3D spatial location and orientation of the amino acid in a frame of reference of the first amino acid chain.

In some implementations, the structure parameters for the first amino acid chain in the protein include global structure parameters that define a 3D spatial location and orientation of the first amino acid chain in a frame of reference of the protein.

In some implementations, applying the symmetrical expansion transformation to the current structure parameters for the first amino acid chain to generate respective current structure parameters for each other amino acid chain in the protein comprises, for each other amino acid chain in the protein: generating the global structure parameters for the other amino acid chain by applying a predefined transformation to the global structure parameters for the first amino acid chain, wherein the predefined transformation depends on: (i) a number of amino acid chains in the protein, and (ii) the symmetry group; and determining amino acid structure parameters for the amino acids in the other amino acid chain that match the amino acid structure parameters for the amino acids in the first amino acid chain.

In some implementations, the symmetry group is a cyclic symmetry group, a dihedral symmetry group, or a cubic symmetry group.

In some implementations, the input processed by the folding neural network further comprises: (i) a respective initial amino acid embedding for each amino acid in the first amino acid chain, and (ii) an initial global embedding of the first amino acid chain.

In some implementations, the operations performed by each update block further comprise receiving a respective current amino acid embedding for each amino acid in the first amino acid chain and a current global embedding of the first amino acid chain; and processing the current structure parameters for the amino acid chains in the protein to update the current structure parameters for the first amino acid chain comprises: updating the current amino acid embeddings and the current global embedding for the first amino acid chain based on the current structure parameters for the amino acid chains in the protein; and updating the current structure parameters for the first amino acid chain based on updated amino acid embeddings and the updated global embedding for the first amino acid chain.

In some implementations, updating the current amino acid embeddings for the first amino acid chain based on the current structure parameters for the amino acid chains in the protein comprises: determining, for each other amino acid chain in the protein, a respective current amino acid embedding for each amino acid in the other amino acid chain based on the current amino acid embedding of a corresponding amino acid in the first amino acid chain; and updating the current amino acid embeddings and the current global embedding for the first amino acid chain using attention over the current amino acid embeddings for the amino acid chains, wherein the attention over the current amino acid embeddings for the amino acid chains is conditioned on the current structure parameters for the amino acid chains.

In some implementations, updating the current global embedding for the first amino acid chain using attention over the current amino acid embeddings for the amino acid chains comprises: determining, for each amino acid in each amino acid chain, a respective attention weight between the current global embedding for the first amino acid chain and the current amino acid embedding for the amino acid based at least in part on: (i) the global structure parameters for the first amino acid chain, and (ii) the amino acid structure parameters for the amino acid and the global structure parameters for the amino acid chain of the amino acid; and updating the current global embedding for the first amino acid chain based on: (i) the attention weights, and (ii) the current amino acid embeddings for the amino acid chains.

In some implementations, for each amino acid in each amino acid chain, determining the attention weight between the current global embedding for the first amino acid chain and the current amino acid embedding for the amino acid comprises: generating a geometric query embedding corresponding to the current global embedding for the first amino acid chain, comprising: processing the current global embedding for the first amino acid chain using one or more neural network layers to generate a 3D embedding; rotating and translating the 3D embedding into a frame of reference of the protein using the global structure parameters for the first amino acid chain; generating a geometric key embedding corresponding to the amino acid, comprising: processing the current amino acid embedding of the amino acid using one or more neural network layers to generate a 3D embedding; and rotating and translating the 3D embedding into the frame of reference of the protein using the amino acid structure parameters for the amino acid and the global structure parameters for the amino acid chain of the amino acid; and determining the attention weight based on a spatial distance between: (i) the geometric query embedding corresponding to the current global embedding for the first amino acid chain, and (ii) the geometric key embedding corresponding to the amino acid.

In some implementations, updating the current structure parameters for the first amino acid chain based on the updated amino acid embeddings and the updated global embedding for the first amino acid chain comprises: for each amino acid in the first amino acid chain, updating the amino acid structure parameters for the amino acid based on the updated amino acid embedding for the amino acid; and updating the global structure parameters for the first amino acid chain based on the updated global embedding for the first amino acid chain.

Particular embodiments of the subject matter described in this specification can be implemented so as to realize one or more of the following advantages

This specification describes a system for predicting the structure of a protein that includes multiple identical amino acid chains and that is expected to fold into a structure that is symmetrical with respect to a symmetry group (e.g., a cyclic, dihedral, or cubic symmetry group). In particular, the system predicts the structure of the protein using a neural network that iteratively refines a current predicted structure of the protein while explicitly enforcing that, at each iteration, the current predicted structure of the protein at the iteration is symmetrical with respect to the symmetry group. Explicitly enforcing the known symmetry of the protein structure throughout the process of iteratively refining predicted protein structure can greatly increase the accuracy of structure predictions made by the neural network.

To explicitly enforce protein structure symmetry, the neural network can internally represent the current predicted protein structure as a function of the predicted structure of a single acid chain in the protein. The neural network directly updates the predicted structure of only the single amino acid chain, while the remainder of the protein structure is indirectly updated as a result of being defined as a function of the predicted structure of the single amino acid chain. This enables the neural network to perform fewer operations and therefore consume fewer computational resources (e.g., memory and computing power), e.g., compared to a neural network that performs operations to independently update the predicted structure of each amino acid chain in the protein.

The structure of a protein determines the biological function of the protein. Therefore, determining protein structures may facilitate understanding life processes (e.g., including the mechanisms of many diseases) and designing proteins (e.g., as drugs, or as enzymes for industrial processes). For example, which molecules (e.g., drugs) will bind to a protein (and where the binding will occur) depends on the structure of the protein. Since the effectiveness of drugs can be influenced by the degree to which they bind to proteins (e.g., in the blood), determining the structures of different proteins may be an important aspect of drug development. However, determining protein structures using physical experiments (e.g., by x-ray crystallography) can be time-consuming and very expensive. Therefore, the protein prediction system described in this specification may facilitate areas of biochemical research and engineering which involve proteins (e.g., drug development).

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates a symmetrical expansion block processing chain structure parameters for a first amino acid chain in a protein to generate structure parameters for the other amino acid chains in the protein that collectively define a protein structure that is symmetrical with respect to the C4 symmetry group.

FIG. 2 shows an example architecture of a folding neural network.

FIG. 3 shows an example protein structure prediction system.

FIG. 4 shows an example embedding system.

FIG. 5 shows an example architecture of an embedding neural network.

FIG. 6 shows an example architecture of an update block of an embedding neural network.

FIG. 7 shows an example architecture of a MSA update block.

FIG. 8 shows an example architecture of a pair update block.

FIG. 9 shows an example embedding expansion system.

FIG. 10 is a flow diagram of an example process for predicting a structure of a protein that includes multiple amino acid chains.

Like reference numbers and designations in the various drawings indicate like elements.

### DETAILED DESCRIPTION

This specification describes a protein structure prediction system (“system”) that is configured to predict the structures of proteins that are composed of multiple amino acid chains. The amino acid chains may be identical, or close to identical. Such proteins include, but are not limited to, protein complexes. Amino acids chains are said to be “identical” if are composed of the same sequence of amino acids.

In many cases, proteins that are composed of multiple, e.g. identical, amino acid chains fold into a symmetrical protein structure. A protein structure is said to be symmetrical with respect to a symmetry group (i.e., a class of transformation operations) if the protein structure is invariant under transformation operations from the symmetry group, i.e., if applying any transformation operation from the symmetry group to the protein structure leaves the orientation of the protein structure effectively unchanged. The transformation operations in a symmetry group may include, e.g., rotations and reflections about a plane.

Examples of symmetry groups include cyclic symmetry groups (e.g., the C2 symmetry group, the C3 symmetry group, the C4 symmetry group, etc.), dihedral symmetry groups (e.g., the D2 symmetry group, the D3 symmetry group, the D4 symmetry group, etc.), and cubic symmetry groups. For example, the C4 symmetry group refers to the class of transformations defined by rotations in multiples of 90 degrees (e.g., 90 degrees, 180 degrees, 270 degrees, 360 degrees, etc.) about an axis.

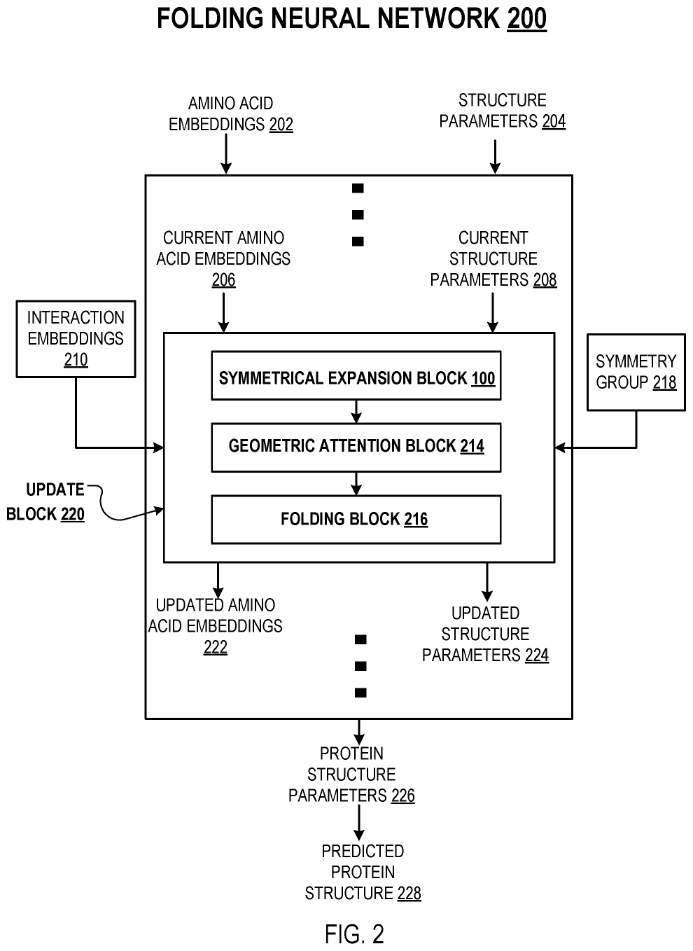

As part of predicting the structure of a symmetrical protein composed of multiple, e.g. identical amino acid chains, the protein structure prediction system uses a symmetrical expansion block 100, i.e., as part of a folding neural network that will be described in more detail with reference to FIG. 2.

The symmetrical expansion block 100 is configured to receive a block input that characterizes one amino acid chain in the protein, which for convenience is referred to herein as the “first” amino acid chain of the protein. (The term “first amino acid chain,” as used in this document, serves only as a convenient way to distinguish one of the amino acids chains of the protein from the others. Any of the amino acid chains of the protein can be the “first amino acid chain”). The block input to the symmetrical expansion block includes: (i) structure parameters for the first amino acid chain (i.e., the “chain structure parameters” 104), and (ii) data identifying a symmetry group of the protein complex (i.e., such that the protein structure is symmetrical with respect to the symmetry group).

The chain structure parameters 104 for the first amino acid chain of the protein complex can include: (i) respective “amino acid” structure parameters for each amino acid in the first amino acid chain, and (ii) “global” structure parameters for the first amino acid chain.

The amino acid structure parameters for each amino acid in an amino acid chain can include, e.g., location parameters for the amino acid that define 3-D spatial location of a specified atom in the amino acid, and rotation parameters that specify the orientation of the amino acid. The location parameters for an amino acid can be represented, e.g., by (x, y, z) Cartesian coordinates, and the rotation parameters for an amino acid can be represented, e.g., by an orthonormal 3×3 matrix with determinant equal to 1, as described above.

The global structure parameters for an amino acid chain can include “global” location parameters and “global” rotation parameters for the amino acid chain. Global location parameters for an amino acid chain can be represented, e.g., by (x, y, z) Cartesian coordinates, and global rotation parameters for an amino acid chain can be represented, e.g., by an orthonormal 3×3 matrix with determinant equal to 1.

The amino acid structure parameters and the global structure parameters for an amino acid chain collectively define the (predicted) position and orientation of the amino acids in the amino acid chain in the structure of the protein complex. For example, the spatial position of each amino acid can be obtained by summing the location parameters for the amino acid and the global location parameters for the amino acid chain. The orientation of each amino acid can be obtained by composing (i.e., matrix multiplying) rotation matrices representing the amino acid rotation parameters and the global rotation parameters for the amino acid chain.

Generally, the amino acid structure parameters for an amino acid chain can be understood as defining the spatial positions and orientations of the amino acids in the amino acid chain in a local frame of reference of the amino acid chain. The global structure parameters for the amino acid chain can be understood as defining translation and rotation operations that move the entire amino acid chain into its position in the global frame of reference of the protein structure, i.e., relative to the other amino acid chains.

The data identifying the symmetry group 106 of the protein can be represented, e.g., by a one-hot vector.

The symmetrical expansion block 100 processes the data defining the symmetry group 106 of the protein 110 and the chain structure parameters 104 for the first amino acid chain in the protein to generate chain structure parameters for each other amino acid chain in the protein (i.e., the “protein structure parameters” 108). In particular, the symmetrical expansion block 100 generates chain structure parameters for the other amino acid chains in the protein such that the chain structure parameters for the amino acid chains collectively define a structure of the protein complex that is symmetrical with respect to the symmetry group 106.

Generally, the amino acid structure parameters are the same for each amino acid chain in the protein, i.e., because the structure of each amino acid chain is the same in the local frame of reference of the amino acid chain. However, the global structure parameters are different for each amino acid chain in the protein.

The symmetrical expansion block 100 can generate respective global structure parameters for each other amino acid chain by applying a “symmetrical expansion transformation” to the global structure parameters of the first amino acid chain. The symmetrical expansion transformation is a predefined function that, when applied to the global structure parameters of the first amino acid chain, generates respective global structure parameters for each other amino acid chain in the protein complex, such that the resulting protein complex structure is symmetrical with respect to the symmetry group 106. Generally, the symmetrical expansion block 100 uses a different predefined symmetrical expansion transformation depending on: (i) the symmetry group 106 of the protein structure, and (ii) the number of amino acid chain chains in the protein.

In one example, if the symmetry group 106 is a C2 symmetry group and the protein complex has two amino acid chains, then the symmetrical expansion transformation can generate the global structure parameters for the other amino acid chain by applying a 180 degree rotation operation to the global rotation parameters of the first amino acid chain. In this example, the global location parameters may be the same for both the amino acid chains in the protein complex.

As another example, if the symmetry group 106 is a C4 symmetry group and the protein complex has four amino acid chains, then the symmetrical expansion transformation can generate the global structure parameters for the three other amino acid chains by applying a 90, 180, and 270 degree rotation operations to the global rotation parameters of the first amino acid chain. In this example, the global location parameters may be the same for all the amino acid chains in the protein complex.

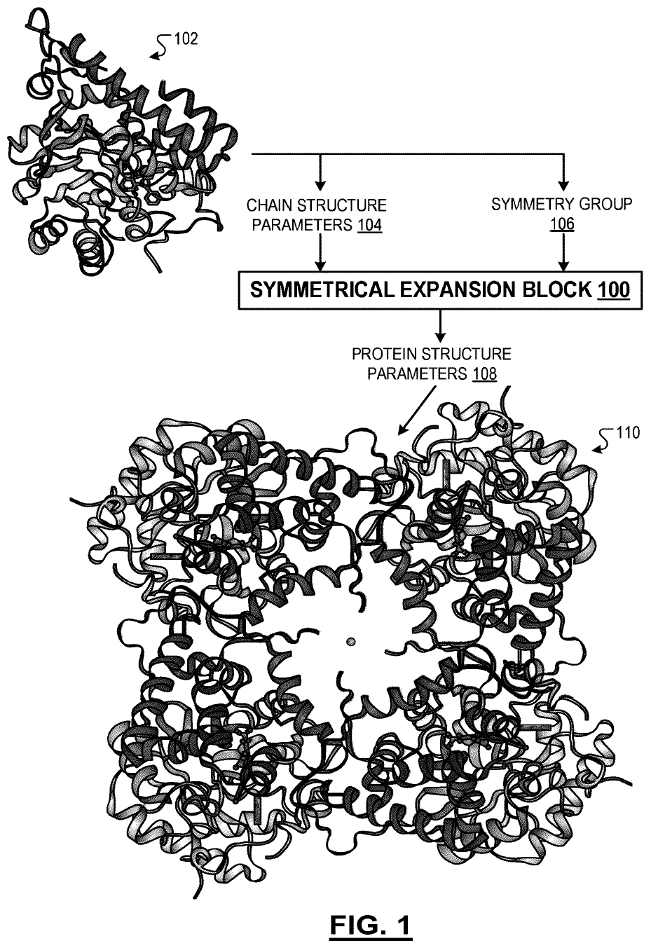

FIG. 1 illustrates the symmetrical expansion block 100 processing the chain structure parameters 104 for the first amino acid chain 102 to generate structure parameters for the other amino acid chains to collectively define a symmetrical protein complex 110 with respect to the C4 symmetry group.

The protein structure prediction system can predict the structure of a protein using a folding neural network that iteratively refines the chain structure parameters for the first amino acid chain in the protein. The folding neural network can use symmetrical expansion blocks, as described with reference to FIG. 1, to define the entire structure of the symmetrical protein as a function of the chain structure parameters of the first amino acid chain in the protein. Using symmetrical expansion blocks enables the folding neural network to explicitly enforce the known symmetry of the protein structure being predicted, thereby improving the accuracy of the protein structure prediction.

FIG. 2 shows an example architecture of a folding neural network 200 that generates protein structure parameters 226 defining a predicted structure 228 of a symmetrical protein that includes multiple identical amino acid chains. That is, the predicted structure 228 of the protein can be defined by a set of structure parameters 226 that collectively define a predicted three-dimensional structure of the protein after the protein undergoes protein folding.

The protein structure prediction system provides an input to the folding neural network 200 that includes: (i) a respective amino acid embedding 202 for each amino acid in a first amino acid chain of the protein, (ii) chain structure parameters 204 for the first amino acid chain, (iii) data defining a symmetry group 218 of the protein, and (iv) a set of interaction embeddings 210.

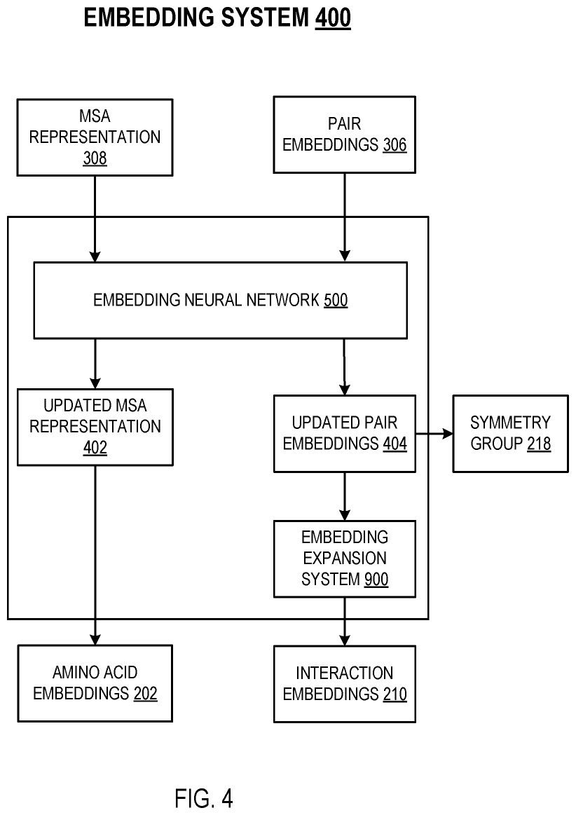

The protein structure prediction system can generate the amino acid embeddings for the first amino acid chain using an embedding system that will be described in more detail with reference to FIG. 4.

The structure parameters 204 for the first amino acid chain include: (i) respective amino acid structure parameters for each amino acid in the first amino acid chain, and (ii) global structure parameters for the first amino acid chain, as described above with reference to FIG. 1. The protein structure prediction system can initialize the amino acid structure parameters for the first amino acid chain as default (predefined) values, e.g., the location parameters for each amino acid can be initialized to the origin (e.g., [0,0,0]), and the rotation parameters for each amino acid can be initialized to a 3×3 identity matrix. The protein structure prediction system can similarly initialize the global structure parameters for the first amino acid chain to default values. Optionally, the protein structure prediction system can generate the initial values of the global structure parameters for the first amino acid chain by processing the interaction embeddings using one or more neural network layers, e.g., fully-connected neural network layers.

The data defining the symmetry group 218 of the protein structure can be represented, e.g., by a one-hot vector. The protein structure prediction system can internally predict the symmetry group 218 of the protein structure, as will be described in more detail with reference to FIG. 4.

The set of interaction embeddings 210 can be represented as a 2-D array of interaction embeddings having N_{AA} rows (i.e., where N_{AA} is the number of amino acids in the first amino acid chain) and N_{AA}·N_{C} columns (i.e., where N_{C} is the number of amino acid chains in the protein). That is, the number of columns of the 2-D array of interaction embeddings can be equal to the total number of amino acids in the protein.

Generally, each interaction embedding 210 corresponds to a respective pair of amino acids in the protein and characterizes the relationship between the pair of amino acids in the protein, e.g., by encoding information characterizing the spatial distance between the pair of amino acids in the structure of the protein. For example, the amino acids in the first amino acid chain can be indexed from {1, . . . , N_{AA}}, the amino acids in the entire protein complex (i.e., including all the amino acid chains) can be indexed from {1, . . . , N_{AA}·N_{C}}, and the interaction embedding at position (i,j) in the array can characterize the relationship between amino acid i in the first amino acid chain and amino acid j in the protein complex. The protein structure prediction system can generate the interaction embeddings using an embedding system, as will be described in more detail with reference to FIG. 4.

In addition to obtaining respective amino acid embeddings 202 for the amino acids in the first amino acid chain, the folding neural network 200 obtains a “global” embedding for the first amino acid chain. For example, the folding neural network 200 can generate (i.e., initialize) the global embedding for the first amino acid chain as the result of processing the interaction embeddings 210 by one or more neural network layers, e.g., fully connected neural network layers.

To generate the protein structure parameters 226 defining the predicted protein structure 228, the folding neural network 200 can repeatedly update the current values of the amino acid embeddings 206, the current values of the structure parameters 208 for the first amino acid chain, i.e., starting from their initial values, and the current global embedding for the first amino acid chain. More specifically, the folding neural network 200 includes a sequence of update blocks 220, where each update block 220 is configured to update the current amino acid embeddings 206 for the first amino acid chain (i.e., to generate updated amino acid embeddings 222 for the first amino acid chain), to update the current structure parameters 208 for the first amino acid chain (i.e., to generate updated structure parameters 224 for the first amino acid chain), and to update the current global embedding for the first amino acid chain. The folding neural network 200 may include other neural network layers or blocks in addition to the update blocks, e.g., that may be interleaved with the update blocks.

Each update block 220 can include: (i) a symmetrical expansion block 100, (ii) a geometric attention block 214, and (ii) a folding block 216, each of which will be described in more detail next.

The symmetrical expansion block 100 processes the current structure parameters 208 for the first amino acid chain and the data identifying the symmetry group 218 of the protein to generate current structure parameters for every other amino acid chain in the protein. In particular, the symmetrical expansion block 100 generates current structure parameters for the other amino acid chains in the protein such that the current structure parameters for the amino acid chains collectively define a protein structure that is symmetrical with respect to the symmetry group 218, as described in more detail with reference to FIG. 1.

In addition to generating current chain structure parameters for the other amino acid chains in the protein, the update block associates each of the other amino acid chains with respective amino acid embeddings. More specifically, the update block associates each amino acid in each of the other amino acid chains with a respective amino acid embedding. In particular, for each other amino acid chain, the update block 220 associates the amino acid at position i in the other amino acid chain with the current amino acid embedding for the amino acid at position i in the first amino acid chain. That is, the update block “tiles” the amino acid embeddings for the amino acids in the first amino acid chain across the amino acids in each of the other amino acid chains.

The geometric attention block 214 and the folding block 216 jointly update the amino acid structure parameters and the global structure parameters for the first amino acid chain using geometric attention operations, as will be described in more detail next.

To implement the geometric attention operation, the geometric attention block 214 determines a respective “symbolic query” embedding, “symbolic key” embedding, and “symbolic value” embedding for each amino acid in the first amino acid chain. For example, the geometric attention block 214 can generate the symbol query embedding qᵢ, the symbolic key embedding kᵢ, and the symbolic value embedding vᵢ for amino acid i by processing the corresponding amino acid embedding hᵢ:

qᵢ=Linear(hᵢ) (1)

kᵢ=Linear(hᵢ) (2)

vᵢ=Linear(hᵢ) (3)

where Linear(·) refers to linear layers having independent learned parameter values.

The geometric attention block 214 associates each amino acid of each of the other amino acid chains with the symbolic key embedding and the symbolic value embedding for the corresponding amino acid in the first amino acid chain. In particular, for each other amino acid chain, the geometric attention block 214 associates the amino acid at position i in the other amino acid chain with the symbolic key embedding and symbolic value embedding for the amino acid at position i in the first amino acid chain. That is, the update block tiles the symbolic key embeddings and the symbolic value embeddings for the amino acids in the first amino acid chain across the amino acids of each of the other amino acid chains.

The geometric attention block 214 also generates a “geometric query” embedding, “geometric key” embedding, and “geometric value” embedding for each amino acid in each amino acid chain in the protein. The geometric query, geometric key, and geometric value embeddings for each amino acid are each 3-D points that are initially generated in a local reference frame of the amino acid, and then rotated and translated into a global reference frame of the protein using the structure parameters corresponding to the amino acid and the global structure parameters for the amino acid chain of the amino acid. For example, the geometric attention block 214 can generate the geometric query embedding qᵢ^{p}, geometric key embedding kᵢ^{p}, and geometric value embedding vᵢ^{p} for amino acid i by processing the corresponding amino acid embedding hᵢ:

qᵢ^{p}=(Rᵢ×R_{g})·Linearₚ(hᵢ)+(tᵢ+t_{g}) (4)

kᵢ^{p}=(Rᵢ×R_{g})·Linearₚ(hᵢ)+(tᵢ+t_{g}) (5)

vᵢ^{p}=(Rᵢ×R_{g})·Linearₚ(hᵢ)+(tᵢ+t_{g}) (6)

where Linearₚ(·) refers to linear layers having independent learned parameter values that project hᵢ to a 3-D point (the superscript p indicates that the quantity is a 3-D point), Rᵢ denotes the rotation matrix specified by the rotation parameters for amino acid i, R_{g} denotes the rotation matrix specified by the global rotation parameters for the amino acid chain of amino acid i, × denotes matrix multiplication, tᵢ denotes the location parameters for amino acid i, and t_{g} denotes the global location parameters for the amino acid chain of amino acid i.

To update the amino acid embedding for amino acid i in the first amino acid chain, the geometric attention block 214 can generate attention weights [aⱼ]ⱼ₌₁^{M}, where M is the total number of amino acids in the protein and aⱼ is the attention weight between amino acid i and amino acid j, as:

$$\\ \begin{matrix}{[ {a}_{j} ]}_{j = 1}^{M} = softmax ( {[ \frac{{q}_{i} \cdot {k}_{j}}{\sqrt{m}} + α {\vert {q}_{i}^{{}^{} p} - {k}_{j}^{{}^{} p} \vert}_{2}^{2} + ( {b}_{i , j} \cdot w ) ]}_{j = 1}^{M} ) & ( 7 )\end{matrix}$$

where qᵢ denotes the symbolic query embedding for amino acid i, kⱼ denotes the symbolic key embedding for amino acid j, m denotes the dimensionality of qᵢ and kⱼ^{p}, α denotes a learned parameter, qᵢ^{p} denotes the geometric query embedding for amino acid i, kⱼ^{p} denotes the geometry key embedding for amino acid j, |·|₂ is an L₂ norm, b_{i,j} is the interaction embedding 210 at position (i,j) in the 2-D array of interaction embeddings, and w is a learned weight vector (or some other learned projection operation).

Generally, the interaction embedding for a pair of amino acids implicitly encodes information relating the relationship between the amino acids in the pair, e.g., the distance between the amino acids in the pair. By determining the attention weight between amino acid i and amino acid j based in part on the interaction embedding for amino acids i and j, the folding neural network 200 enriches the attention weights with the information from the interaction embeddings and thereby improves the accuracy of the predicted folding structure.

After generating the attention weights for amino acid embedding hᵢ for amino acid i in the first amino acid chain, the geometric attention block 214 uses the attention weights to update the amino acid embedding hᵢ. In particular, the geometric attention block 214 uses the attention weights to generate a “symbolic return” embedding and a “geometric return” embedding, and then updates the amino acid embedding using the symbolic return embedding and the geometric return embedding. The geometric attention block 214 can generate the symbolic return embedding oᵢ for amino acid i, e.g., as:

$$\\ \begin{matrix}{o}_{i} = {\sum }_{j} {a}_{j} {v}_{j} & ( 8 )\end{matrix}$$

where [aⱼ]ⱼ₌₁^{M} denote the attention weights (e.g., defined with reference to equation (7)), j indexes all the amino acids in the protein, and each vⱼ denotes the symbolic value embedding for amino acid j. The geometric attention block 214 may generate the geometric return embedding oᵢ^{p} for amino acid i, e.g., as:

$$\\ \begin{matrix}{o}_{i}^{{}^{} p} = {( {R}_{i} \times {R}_{g} )}^{- 1} \cdot ( {\sum }_{j} {a}_{j} {v}_{j}^{{}^{} p} - ( {t}_{i} + {t}_{g} ) ) & ( 9 )\end{matrix}$$

where the geometric return embedding oᵢ^{p} is a 3-D point, [aⱼ]ⱼ₌₁^{M} denote the attention weights (e.g., defined with reference to equation (7)), j indexes all the amino acids in the protein, Rᵢ is the rotation matrix specified by the rotation parameters for amino acid i in the first amino acid chain, R_{g} is the rotation matrix specified by the global rotation parameters for the first amino acid chain, tᵢ are the location parameters for amino acid i in the first amino acid chain, and t_{g} are the global rotation parameters for the first amino acid chain. It can be appreciated that the geometric return embedding is initially generated in the global reference frame of the protein, and then rotated and translated to a local reference frame of amino acid i.

The geometric attention block 214 can update the amino acid embedding hᵢ for amino acid i in the first amino acid chain using the corresponding symbolic return embedding oᵢ (e.g., generated in accordance with equation (8)) and geometric return embedding oᵢ^{p} (e.g., generated in accordance with equation (9)), e.g., as:

hᵢ^{next}=LayerNorm(hᵢ+Linear(oᵢ, oᵢ^{p}, |oᵢ^{p}|)) (10)

where hᵢ^{next} is the updated amino acid embedding for amino acid i, |·| is a norm, e.g., an L₂ norm, and LayerNorm( ) denotes a layer normalization operation, e.g., as described with reference to: J. L. Ba, J. R. Kiros, G. E. Hinton, “Layer Normalization,” arXiv:1607.06450 (2016).

Updating the amino acid embeddings 206 using concrete 3-D geometric embeddings, e.g., as described with reference to equations (4)-(6), enables the geometric attention block 214 to reason about 3-D geometry in updating the amino acid embeddings. Moreover, each update block updates the amino acid embeddings and the structure parameters in a manner that is invariant to rotations and translations of the overall protein structure. For example, applying the same global rotation and translation operation to the initial structure parameters provided to the folding neural network 200 would cause the folding neural network 200 to generate a predicted structure that is globally rotated and translated in the same way, but otherwise the same. Therefore, global rotation and translation operations applied to the initial structure parameters would not affect the accuracy of the predicted protein structure generated by the folding neural network 200 starting from the initial structure parameters. The rotational and translational invariance of the representations generated by the folding neural network 200 facilitates training, e.g., because the folding neural network 200 automatically learns to generalize across all rotations and translations of protein structures.

The updated amino acid embeddings for the first amino acid chain may be further transformed by one or more additional neural network layers in the geometric attention block 214, e.g., linear neural network layers, before being provided to the folding block 216.

In addition to updating the amino acid embeddings for the amino acids in the first amino acid chain, the geometric attention block 214 updates the global embedding for the first amino acid chain.

To update the global embedding for the first amino acid chain, the geometric attention block 214 processes the global embedding 11, to generate a corresponding symbolic query embedding q_{g}, e.g., as:

q_{g}=Linear(h_{g})

where Linear(·) refers to linear neural network layers. The geometric attention block 214 also processes the global embedding h_{g} to generate a corresponding geometric query embedding q_{g}^{p}, e.g., as:

q_{g}^{p}=R_{g}·Linearₚ(h_{g})+t_{g}

where Linearₚ(·) refers to linear neural network layers that project h_{g} to a 3-D point (the superscript p indicates that the quantity is a 3-D point), R_{g} denotes the rotation matrix specified by the global rotation parameters for the first amino acid chain, and t_{g} denotes the global location parameters for the first amino acid chain.

The geometric attention block 214 uses the symbolic query embedding and the geometric query embedding for the global embedding of the first amino acid chain to generate attention weights [aⱼ^{g}]ⱼ₌₁^{M}, where M is the total number of amino acids in the protein and aⱼ^{g} is the attention weight between the global embedding and the amino acid embedding for amino acid j, as:

$$\\ {[ {a}_{j}^{{}^{} g} ]}_{j = 1}^{M} = softmax ( {[ \frac{{q}_{g} \cdot {k}_{j}}{\sqrt{m}} + α {\vert {q}_{g}^{{}^{} p} - {k}_{j}^{{}^{} p} \vert}_{2}^{2} ]}_{j = 1}^{M} )$$

where q_{g} denotes the symbolic query embedding for the global embedding of the first amino acid chain, kⱼ denotes the symbolic key embedding for amino acid j, m denotes the dimensionality of q_{g} and kⱼ, a denotes a learned parameter, q_{g}^{p} denotes the geometric query embedding for the global embedding of the first amino acid chain, kⱼ^{p} denotes the geometry key embedding for amino acid j, and |·|₂ is an L₂ norm.

After generating the attention weights for the global embedding of the first amino acid chain, the geometric attention block 214 uses the attention weights to update the global embedding. In particular, the geometric attention block 214 uses the attention weights to generate a “symbolic return” embedding and a “geometric return” embedding, and then updates the global embedding using the symbolic return embedding and the geometric return embedding. The geometric attention block 214 can generate the symbolic return embedding o_{g} for the global embedding, e.g., as:

$$\\ {o}_{g} = {\sum }_{j} {a}_{j}^{{}^{} g} {v}_{j}$$

where [aⱼ^{g}]ⱼ₌₁^{M} denote the attention weights between the global embedding and the amino acid embeddings, j indexes all the amino acids in the protein, and each vⱼ denotes the symbolic value embedding for amino acid j. The geometric attention block 214 may generate the geometric return embedding o_{g}^{p} for the global embedding, e.g., as:

$$\\ {o}_{g}^{{}^{} p} = {( {R}_{g} )}^{- 1} \cdot ( {\sum }_{j} {a}_{j}^{{}^{} g} {v}_{j}^{{}^{} p} - {t}_{g} )$$

where the geometric return embedding o_{g}^{p} is a 3-D point, [aⱼ^{g}]ⱼ₌₁^{M} denote the attention weights between the global embedding and the amino acid embeddings, j indexes all the amino acids in the protein, R_{g} is the rotation matrix specified by the global rotation parameters for the first amino acid chain, and t_{g} are the global rotation parameters for the first amino acid chain.

The geometric attention block 214 can update the global embedding of the first amino acid chain using the corresponding symbolic return embedding o_{g} and geometric return embedding o_{g}^{p}, e.g., as:

h_{g}^{next}=LayerNorm(h_{g}+Linear(g_{g}, o_{g}^{p}, |o_{g}^{p}|)) (10)

where h_{g}^{next} is the updated global embedding for the first amino acid chain, |·| is a norm, e.g., an L₂ norm, and LayerNorm(·) denotes a layer normalization operation.

After the geometric attention block 214 updates the amino acid embeddings 206 for the amino acids in the first amino acid chain and the global embedding of the first amino acid chain, the folding block 216 updates the current structure parameters 208 for the first amino acid chain using the updated amino acid embeddings 222 and the updated global embedding.

For example, the folding block 216 may update the current location parameters tᵢ for amino acid i in the first amino acid chain as:

tᵢ^{net}=tᵢ+Linear(hᵢ^{next}) (11)

where tᵢ^{next} are the updated location parameters, Linear(·) denotes a linear neural network layer, and hᵢ^{next} denotes the updated amino acid embedding for amino acid i.

In another example, the folding block 216 may update the current global location parameters t_{g} for the first amino acid chain as:

t_{g}^{next}=t_{g}+Linear(h_{g}^{next})

where t_{g}^{next} are the updated global location parameters, Linear(·) denotes a linear neural network layer, and h_{g}^{next} denotes the updated global embedding for the first amino acid chain.

In another example, the rotation parameters Rᵢ for amino acid i in the first amino acid chain may specify a rotation matrix, and the folding block 216 may update the current rotation parameters Rᵢ as:

wᵢ=Linear(hᵢ^{next}) (12)

Rᵢ^{next}=Rᵢ·QuaternionToRotation(1+wᵢ) (13)

where wᵢ is a three-dimensional vector, Linear(·) is a linear neural network layer, hᵢ^{next} is the updated amino acid embedding for amino acid i, 1+wᵢ denotes a quaternion with real part 1 and imaginary part wᵢ, and QuaternionToRotation() denotes an operation that transforms a quaternion into an equivalent 3×3 rotation matrix. Updating the rotation parameters using equations (12)-(13) ensures that the updated rotation parameters define a valid rotation matrix, e.g., an orthonormal matrix with determinant one.

In another example, the global rotation parameters R_{g} for the first amino acid chain may specify a rotation matrix, and the folding block 216 may update the current global rotation parameters R_{g} as:

w_{g}=Linear(h_{g}^{next})

R_{g}^{next}=R_{g}·QuaternionToRotation(1+w_{g})

where w_{g} is a three-dimensional vector, Linear(·) is a linear neural network layer, h_{g}^{next} is the updated global embedding, 1+w_{g} denotes a quaternion with real part 1 and imaginary part w_{g}, and QuaternionToRotation(·) denotes an operation that transforms a quaternion into an equivalent 3×3 rotation matrix.

The operations of the geometric attention block 214 and the folding block 216, as described above, jointly update both: (i) the structure parameters for the amino acids in the first amino acid chain, and (ii) the global structure parameters for the first amino acid chain. Updating the structure parameters for the amino acids in the first amino acid chain has the effect of updating the local structure of each amino acid chain in the protein complex. Updating the global structure parameters for the first amino acid chain has the effect of rotating and translating the positions of the amino acids chains in the protein structure relative to one another.

The final update block in the sequence of update blocks of the folding neural network can generate final structure parameters for the first amino acid chain, i.e., including final structure parameters for each amino acid in the first amino acid chain, and final global structure parameters for the first amino acid chain.

The folding neural network 200 can process: (i) the final structure parameters for the first amino acid chain, and (ii) data identifying the symmetry group 218, using a symmetrical expansion block to generate the protein structure parameters 226 that define the predicted symmetrical structure 228 of the protein.

The folding neural network 200 may include any appropriate number of update blocks, e.g., 5 update blocks, 25 update blocks, or 125 update blocks. Optionally, each of the update blocks of the folding neural network may share a single set of parameter values that are jointly updated during training of the folding neural network. Sharing parameter values between the update blocks 220 reduces the number of trainable parameters of the folding neural network and may therefore facilitate effective training of the folding neural network, e.g., by stabilizing the training and reducing the likelihood of overfitting.

During training, a training engine can train the parameters of the protein structure prediction system, including the parameters of the folding neural network 200, based on a structure loss that evaluates the accuracy of the protein structure parameters 226, as will be described in more detail below. In some implementations, the training engine can further evaluate an auxiliary structure loss for one or more of the update blocks 220 that precede the final update block. The auxiliary structure loss for an update block evaluates the accuracy of the protein structure parameters defined by processing the updated structure parameters generated by the update block for the first amino acid chain using a symmetrical expansion block.

Optionally, during training, the training engine can apply a “stop gradient” operation to prevent gradients from backpropagating through certain neural network parameters of each update block, e.g., the neural network parameters used to compute the updated rotation parameters (as described in equations (12)-(13)). Applying these stop gradient operations can improve the numerical stability of the gradients computed during training.

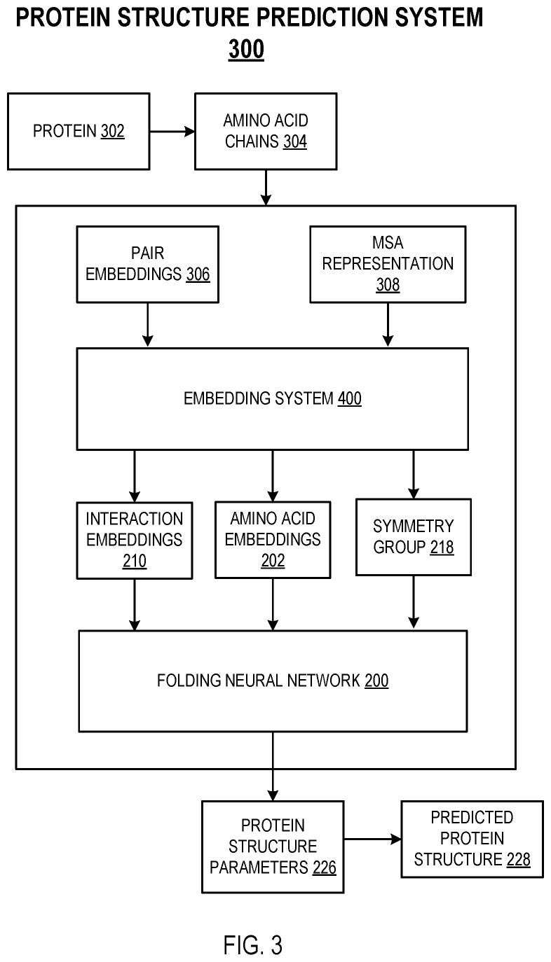

FIG. 3 shows an example protein structure prediction system 300 that includes the folding neural network 200 described with reference to FIG. 2. The protein structure prediction system 300 is an example of a system implemented as computer programs on one or more computers in one or more locations in which the systems, components, and techniques described below are implemented.

The system 300 is configured to generate a set of protein structure parameters 226 that define a predicted symmetrical protein structure 228 of a protein that includes multiple identical amino acid chains 304.

To generate the structure parameters 226 defining the predicted protein structure 228, the system 300 generates: (i) a multiple sequence alignment (MSA) representation 308 for the first amino acid chain in the protein, and (ii) a set of “pair” embeddings 306 for the first amino acid chain in the protein, as will be described in more detail next.

The MSA representation 308 represents a MSA corresponding to the first amino acid chain in the protein. A MSA representation for an amino acid chain can be represented as a M×N array of embeddings (i.e., a 2-D array of embeddings having M rows and N_{AA} columns), where M is the number of MSA sequences in the MSA and N_{AA} is the number of amino acids in the first amino acid chain. Each row of the MSA representation can correspond to a respective MSA sequence. The system can initialize the MSA representation in any appropriate way. For example, the system 300 can initialize the embedding at each position (i,j) in the MSA representation 308 to be a one-hot vector defining the identity of the amino acid at position j in MSA sequence i. Throughout this specification, a “row” of the MSA representation refers to a row of a 2-D array of embeddings defining the MSA representation. Similarly, a “column” of the MSA representation refers to a column of a 2-D array of embeddings defining the MSA representation.

The set of pair embeddings 306 includes a respective pair embedding corresponding to each pair of amino acids in the first amino acid chain of the protein. A pair of amino acids refers to an ordered tuple that includes a first amino acid and a second amino acid in the first amino acid chain, i.e., such that the set of possible pairs of amino acids in the first amino acid chain is given by:

{(Aᵢ, Aⱼ): 1≤i,j≤N_{AA}} (14)

where N_{AA} is the number of amino acids in the first amino acid, i,j∈{1, . . . , N} index the amino acids in the first amino acid chain, Aᵢ is the amino acid in the first amino acid chain indexed by i, and Aⱼ is the amino acid in the first amino acid chain indexed by j. The set of pair embeddings 306 can be represented as a 2-D, N_{AA}×N_{AA} array of pair embeddings, e.g., where the rows of the 2-D array are indexed by i∈{1, . . . , N_{AA}}, the columns of the 2-D array are indexed by j∈{1, . . . , N_{AA}}, and position (i,j) in the 2-D array is occupied by the pair embedding for the pair of amino acids (Aᵢ, Aⱼ).

The system can initialize the pair embeddings, e.g., by applying an outer product mean operation to the MSA representation 308, and identifying the pair embeddings 306 as the result of the outer product mean operation. The outer product mean operation defines a sequence of operations that, when applied to an MSA representation represented as an M×N_{AA} array of embeddings, generates an N_{AA}×N_{AA} array of embeddings, i.e, where N AA is the number of amino acids in the first amino acid chain.

To compute the outer product mean, the system generates a tensor A(·), e.g., given by:

$$\\ A ( res 1 , res 2 , ch 1 , ch 2 ) = \frac{1}{\vert rows \vert} {\sum }_{rows} LeftAct ( row , res 1 , ch 1 ) \cdot RightAct ( row , res 2 , ch 2 )$$

where res1, res2∈{1, . . . , N_{AA}}, ch1, ch2∈{1, . . . , C}, where C is the number of channels in each embedding of the MSA representation, ‘rows’ is the number rows in the MSA representation, LeftAct(row,res1,ch1) is a linear operation (e.g., defined by a matrix multiplication) applied to the channel ch1 of the embedding of the MSA representation located at the row indexed by “row” and the column indexed by “res1”, and RightAct(row, res2, ch2) is a linear operation (e.g., defined by a matrix multiplication) applied to the channel ch2 of the embedding of the MSA representation located at the row indexed by “row” and the column indexed by “res2”. The result of the outer product mean is generated by flattening and linearly projecting the (ch1, ch2) dimensions of the tensor A. Optionally, the system can perform one or more Layer Normalization operations (e.g., as described with reference to Jimmy Lei Ba et al., “Layer Normalization,” arXiv:1607.06450) as part of computing the outer product mean.

The system 300 generates the structure parameters 226 defining the predicted protein structure 228 using both the MSA representation 308 and the pair embeddings 306, because both have complementary properties. The structure of the MSA representation 308 can explicitly depend on the number of amino acid chains in the MSA. Therefore, the MSA representation 308 may be inappropriate for use in directly predicting the protein structure, because the protein structure 228 has no explicit dependence on the number of amino acids chains in the MSAs. In contrast, the pair embeddings 306 characterize relationships between respective pairs of amino acids in the protein 302 and are expressed without explicit reference to the MSAs, and are therefore a convenient and effective data representation for use in predicting the protein structure 228.

The system 300 processes the MSA representation 308 and the pair embeddings 306 using an embedding system 400 to generate the input provided to the folding neural network 200, i.e.: the interaction embeddings 210, the amino acid embeddings 202 for the first amino acid chain, and the symmetry group 218 of the protein.

An example embedding system 400 is described in more detail with reference to FIG. 4.

The system 300 generates a network input for the folding neural network 200 from the interaction embeddings 210, the amino acid embeddings 202, and the symmetry group 218, and processes the network input using the folding neural network 200 to generate the structure parameters 226 defining the predicted protein structure.

A training engine may train the protein structure prediction system 300 from end-to-end to optimize an objective function referred to herein as a structure loss. The training engine may train the system 300 on a set of training data including multiple training examples. Each training example may specify: (i) a training input that includes a MSA representation and pair embeddings for a protein, and (ii) a target protein structure that should be generated by the system 300 by processing the training input. Target protein structures used for training the system 300 may be determined using experimental techniques, e.g., x-ray crystallography or cryo-EM.

The structure loss may characterize a similarity between: (i) a predicted protein structure generated by the system 300, and (ii) the target protein structure that should have been generated by the system.

For example, if the predicted structure parameters define predicted location parameters and predicted rotation parameters for each amino acid in the protein, then the structure loss [symbol] _{structure} may be given by:

$$\\ \begin{matrix}{ℒ}_{structure} = \frac{1}{{N}^{{}^{} 2}} {\sum }_{i , j = 1}^{N} {( 1 - \frac{\vert {t}_{ij} - \vert}{A} )}_{+} & ( 15 )\end{matrix}$$

$$\\ \begin{matrix}{t}_{ij} = {R}_{i}^{{}^{} - 1} ( {t}_{j} - {t}_{i} ) & ( 16 )\end{matrix}$$

$$\\ \begin{matrix}= \tilde{R} {}_{ι}^{{}^{} - 1} ( - ) & ( 17 )\end{matrix}$$

where N is the number of amino acids in the protein, tᵢ denote the predicted location parameters for amino acid i, Rᵢ denotes a 3×3 rotation matrix specified by the predicted rotation parameters for amino acid i, \tilde{t}ᵢ are the target location parameters for amino acid i, \tilde{R}ᵢ denotes a 3×3 rotation matrix specified by the target rotation parameters for amino acid i, A is a constant, Rᵢ⁻¹ refers to the inverse of the 3×3 rotation matrix specified by predicted rotation parameters Rᵢ, \tilde{R}ᵢ⁻¹ refers to the inverse of the 3×3 rotation matrix specified by the target rotation parameters \tilde{R}ᵢ, and (·)₊ denotes a rectified linear unit (ReLU) operation.

The structure loss defined with reference to equations (15)-(17) may be understood as averaging the loss |tᵢⱼ− [symbol] | over each pair of amino acids in the protein. The term tᵢⱼ defines the predicted spatial location of amino acid j in the predicted frame of reference of amino acid i, and [symbol] defines the actual spatial location of amino acid j in the actual frame of reference of amino acid i. These terms are sensitive to the predicted and actual rotations of amino acid i and j, and therefore carry richer information than loss terms that are only sensitive to the predicted and actual distances between amino acids.

As part of evaluating the structure loss, the training determines which amino acid chain of the predicted protein structure corresponds to which amino acid chain of the ground truth protein structure. In some implementations, the training engine computes the structure loss for every possible mapping of the amino acid chains of the predicted protein structure to the amino acid chains of the ground truth protein structure, and uses the minimum of these structure losses to train the system.

Optimizing the structure loss encourages the system 300 to generate predicted protein structures that accurately approximate true protein structures.

In addition to optimizing the structure loss, the training engine may train the system 300 to optimize one or more auxiliary losses. The auxiliary losses may penalize predicted structures having characteristics that are unlikely to occur in the natural world, e.g., based on the bond angles and/or bond lengths of the bonds between the atoms in the amino acids in the predicted structures, or based on the proximity of the atoms in different amino acids in the predicted structures.

The training engine may train the structure prediction system 300 on the training data over multiple training iterations, e.g., using stochastic gradient descent training techniques.

FIG. 4 shows an example embedding system 400. The embedding system 400 is an example of a system implemented as computer programs on one or more computers in one or more locations in which the systems, components, and techniques described below are implemented.

The system 400 processes the MSA representation 308 and the pair embeddings 306 using an embedding neural network 500, in accordance with the values of a set of parameters of the embedding neural network 500, to update the MSA representation 308 and the pair embeddings 306. That is, the embedding neural network 500 processes the MSA representation 308 and the pair embeddings 306 to generate an updated MSA representation 402 and updated pair embeddings 404.

The embedding neural network 500 updates the MSA representation 308 and the pair embeddings 306 by sharing information between the MSA representation 308 and the pair embeddings 306. More specifically, the embedding neural network 500 alternates between updating the current MSA representation 308 based on the current pair embeddings 306, and updating the current pair embeddings 306 based on the current MSA representation 308.

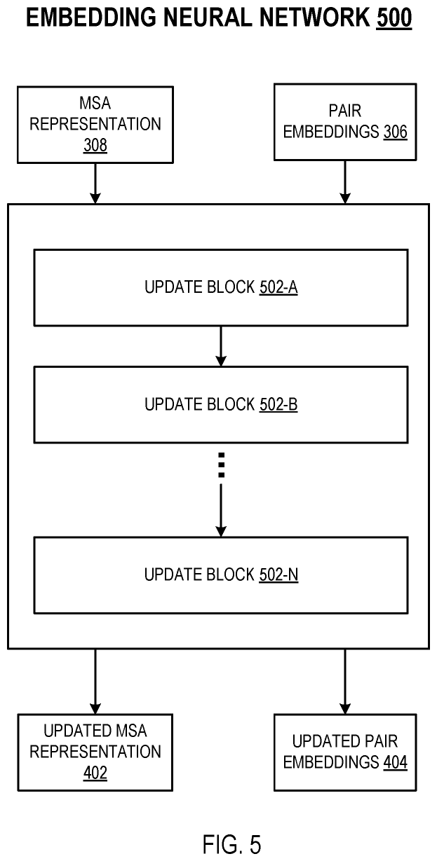

An example architecture of an embedding neural network is described in more detail with reference to FIG. 5.

The embedding system 400 can generate the amino acid embeddings 202 for the amino acids in the first amino acid chain from the updated MSA representation 402. For example, the updated MSA representation 402 can be represented as a 2-D array of embeddings having a number of columns equal to the number of amino acids in the first amino acid chain, where each column is associated with a respective amino acid in the first amino acid chain. The embedding system 400 can generate the initial amino acid embedding for each amino acid in the first amino acid chain by summing (or otherwise combining) the embeddings from the column of the MSA representation 308 that is associated with the amino acid. As another example, the embedding system 400 can generate the initial amino acid embeddings for the amino acids in the first amino acid chain by extracting the embeddings from a row of the MSA representation 308 that corresponds to the amino acid sequence of the first amino acid chain.

To generate the symmetry group 218, the embedding system 400 processes: (i) the updated pair embeddings, and (ii) data identifying the number of amino acid chains in the protein (e.g., as a one-hot vector), using one or more neural network layers to generate a probability distribution over a set of possible symmetry groups. The embedding system 400 can select the symmetry group 218 using the probability distribution, e.g., by sampling a possible symmetry group in accordance with the probability distribution, or by selecting the symmetry group having the highest probability under the probability distribution.

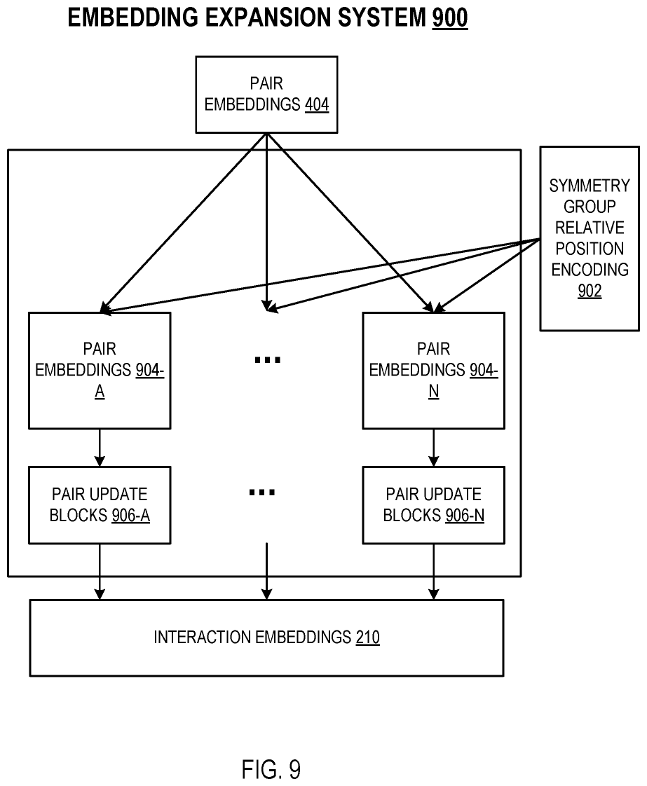

The embedding system processes the updated pair embeddings 404 using an embedding expansion system 400 to generate the interaction embeddings 210. An example embedding expansion system 900 is described in more detail with reference to FIG. 9.

FIG. 5 shows an example architecture of an embedding neural network 500 that is configured to process the MSA representation 308 and the pair embeddings 306 to generate the updated MSA representation 402 and the updated pair embeddings 404.

The embedding neural network 500 includes a sequence of update blocks 502-A-N. Throughout this specification, a “block” refers to a portion of a neural network, e.g., a subnetwork of the neural network that includes one or more neural network layers.

Each update block in the embedding neural network is configured to receive a block input that includes a MSA representation and a pair embedding, and to process the block input to generate a block output that includes an updated MSA representation and an updated pair embedding.

The embedding neural network 500 provides the MSA representation 308 and the pair embeddings 306 included in the network input of the embedding neural network 500 to the first update block (i.e., in the sequence of update blocks). The first update block processes the MSA representation 308 and the pair embeddings 306 to generate an updated MSA representation and updated pair embeddings.

For each update block after the first update block, the embedding neural network 500 provides the update block with the MSA representation and the pair embeddings generated by the preceding update block, and provides the updated MSA representation and the updated pair embeddings generated by the update block to the next update block.

The embedding neural network 500 gradually enriches the information content of the MSA representation 308 and the pair embeddings 306 by repeatedly updating them using the sequence of update blocks 502-A-N.

The embedding neural network 500 may provide the updated MSA representation 402 and the updated pair embeddings 404 generated by the final update block (i.e., in the sequence of update blocks) as the network output.

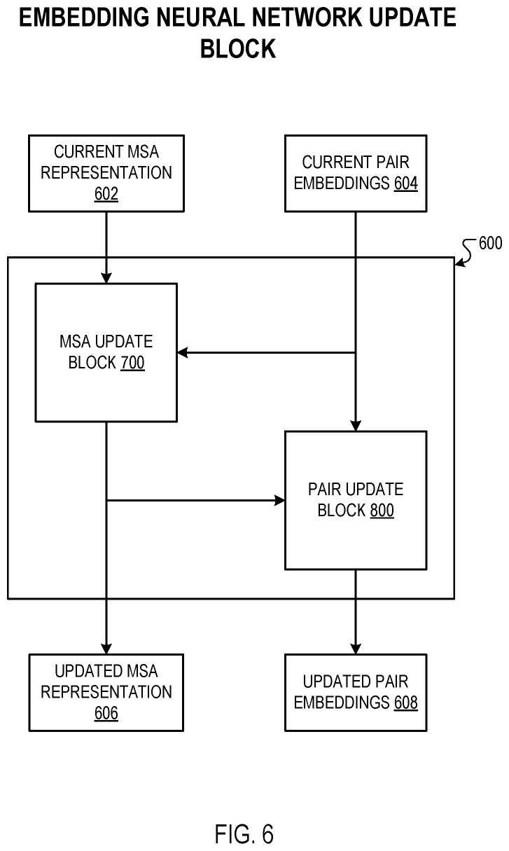

FIG. 6 shows an example architecture of an update block 600 of the embedding neural network 500, i.e., as described with reference to FIG. 5.

The update block 600 receives a block input that includes the current MSA representation 602 and the current pair embeddings 604, and processes the block input to generate the updated MSA representation 606 and the updated pair embeddings 608. The update block 600 includes an MSA update block 700 and a pair update block 800.

The MSA update block 700 updates the current MSA representation 602 using the current pair embeddings 604, and the pair update block 800 updates the current pair embeddings 604 using the updated MSA representation 606 (i.e., that is generated by the MSA update block 700).

Generally, the MSA representation and the pair embeddings can encode complementary information. For example, the MSA representation can encode information about the correlations between the identities of the amino acids in different positions among a set of evolutionarily-related amino acid chains, and the pair embeddings can encode information about the inter-relationships between the amino acids in the protein. The MSA update block 700 enriches the information content of the MSA representation using complementary information encoded in the pair embeddings, and the pair update block 800 enriches the information content of the pair embeddings using complementary information encoded in the MSA representation. As a result of this enrichment, the updated MSA representation and the updated pair embedding encode information that is more relevant to predicting the protein structure.

The update block 600 is described herein as first updating the current MSA representation 602 using the current pair embeddings 604, and then updating the current pair embeddings 604 using the updated MSA representation 606. The description should not be understood as limiting the update block to performing operations in this sequence, e.g., the update block could first update the current pair embeddings using the current MSA representation, and then update the current MSA representation using the updated pair embeddings.

The update block 600 is described herein as including an MSA update block 700 (i.e., that updates the current MSA representation) and a pair update block 800 (i.e., that updates the current pair embeddings). The description should not be understood to limiting the update block 600 to include only one MSA update block or only one pair update block. For example, the update block 600 can include multiple MSA update blocks that update the MSA representation multiple times before the MSA representation is provided to a pair update block for use in updating the current pair embeddings. As another example, the update block 600 can include multiple pair update blocks that update the pair embeddings multiple times using the MSA representation.

The MSA update block 700 and the pair update block 800 can have any appropriate architectures that enable them to perform their described functions.

In some implementations, the MSA update block 700, the pair update block 800, or both, include one or more “self-attention” blocks. As used throughout this document, a self-attention block generally refers to a neural network block that updates a collection of embeddings, i.e., that receives a collection of embeddings and outputs updated embeddings. To update a given embedding, the self-attention block can determine a respective “attention weight” between the given embedding and each of one or more selected embeddings, and then update the given embedding using: (i) the attention weights, and (ii) the selected embeddings. For convenience, the self-attention block may be said to update the given embedding using attention “over” the selected embeddings.

For example, a self-attention block may receive a collection of input embeddings {xᵢ}ᵢ₌₁^{N}^{_{AA}}, where N_{AA} is the number of amino acids in the first amino acid chain, and to update embedding xᵢ, the self-attention block may determine attention weights [a_{i,j}]ⱼ₌₁^{N}^{_{AA}} where a_{i,j} denotes the attention weight between xᵢ and xⱼ, as:

$$\\ \begin{matrix}{[ {a}_{i , j}^{} ]}_{j = 1}^{{N}_{AA}} = softmax {}^{} ( \frac{( {W}_{q} {x}_{i} ) {K}^{{}^{} T}}{c} ) & ( 18 )\end{matrix}$$

$$\\ \begin{matrix}{K}^{{}^{} T} = {[ {W}_{k} {x}_{j} ]}_{j = 1}^{{N}_{AA}} & ( 19 )\end{matrix}$$

where W_{q} and Wₖ are learned parameter matrices, softmax(·) denotes a soft-max normalization operation, and c is a constant. Using the attention weights, the self-attention layer may update embedding xᵢ as:

$$\\ \begin{matrix}{x}_{i} ← {\sum }_{j = 1 .. {N}_{AA}} {a}_{i , j} \cdot ( {W}_{v} {x}_{j} ) & ( 20 )\end{matrix}$$

where Wᵥ is a learned parameter matrix. (W_{q}xᵢ can be referred to as the “query embedding” for input embedding xᵢ, Wₖxⱼ can be referred to as the “key embedding” for input embedding xᵢ, and Wᵥxⱼ can be referred to as the “value embedding” for input embedding xᵢ).

The parameter matrices W_{q} (the “query embedding matrix”), Wₖ (the “key embedding matrix”), and Wᵥ (the “value embedding matrix”) are trainable parameters of the self-attention block. The parameters of any self-attention blocks included in the MSA update block 700 and the pair update block 800 can be understood as being parameters of the update block 600 that can be trained as part of the end-to-end training of the protein structure prediction system 300 described with reference to FIG. 3. Generally, the (trained) parameters of the query, key, and value embedding matrices are different for different self-attention blocks, e.g., such that a self-attention block included in the MSA update block 700 can have different query, key, and value embedding matrices with different parameters than a self-attention block included in the pair update block 800.

In some implementations, the MSA update block 700, the pair update block 800, or both, include one or more self-attention blocks that are conditioned on the pair embeddings, i.e., that implement self-attention operations that are conditioned on the pair embeddings. To condition a self-attention operation on the pair embeddings, the self-attention block can process the pair embeddings to generate a respective “attention bias” corresponding to each attention weight. For example, in addition to determining the attention weights [a_{i,j}]ⱼ₌₁^{N}^{_{AA}} in accordance with equations (18)-(19), the self-attention block can generate a corresponding set of attention biases [b_{i,j}]ⱼ₌₁^{N}^{_{AA}}, where b_{i,j} denotes the attention bias between xᵢ and xⱼ. The self-attention block can generate the attention bias b_{i,j} by applying a learned parameter matrix to the pair embedding h_{i,j}, i.e., for the pair of amino acids in the protein indexed by (i,j).

The self-attention block can determine a set of “biased attention weights” [c_{i,j}]ⱼ₌₁^{N}^{_{AA}}, where c_{i,j} denotes the biased attention weight between xᵢ and xᵢ, e.g., by summing (or otherwise combining) the attention weights and the attention biases. For example, the self-attention block can determine the biased attention weight c_{i,j} between embeddings xᵢ and xⱼ as:

c_{i,j}=a_{i,j}+b_{i,j}

where a_{i,j} is the attention weight between xᵢ and xⱼ and b_{i,j} is the attention bias between xᵢ and xⱼ. The self-attention block can update each input embedding xᵢ using the biased attention weights, e.g.:

$$\\ \begin{matrix}{x}_{i} ← {\sum }_{j = 1 .. N} {c}_{i , j} \cdot ( {W}_{v} {x}_{j} ) & ( 21 )\end{matrix}$$

where Wᵥ is a learned parameter matrix.

Generally, the pair embeddings encode information characterizing the structure of the protein and the relationships between the pairs of amino acids in the structure of the protein. Applying a self-attention operation that is conditioned on the pair embeddings to a set of input embeddings allows the input embeddings to be updated in a manner that is informed by the protein structural information encoded in the pair embeddings. The update blocks of the embedding neural network can use the self-attention blocks that are conditioned on the pair embeddings to update and enrich the MSA representation and the pair embeddings themselves.

Optionally, a self-attention block can have multiple “heads” that each generate a respective updated embedding corresponding to each input embedding, i.e., such that each input embedding is associated with multiple updated embeddings. For example, each head may generate updated embeddings in accordance with different values of the parameter matrices W_{q}, Wₖ, and Wᵥ that are described with reference to equations (18)-(21). A self-attention block with multiple heads can implement a “gating” operation to combine the updated embeddings generated by the heads for an input embedding, i.e., to generate a single updated embedding corresponding to each input embedding. For example, the self-attention block can process the input embeddings using one or more neural network layers (e.g., fully connected neural network layers) to generate a respective gating value for each head. The self-attention block can then combine the updated embeddings corresponding to an input embedding in accordance with the gating values. For example, the self-attention block can generate the updated embedding for an input embedding xᵢ as:

$$\\ \begin{matrix}{\sum }_{k = 1}^{K} {α}_{k} \cdot {x}_{i}^{{}^{} next} & ( 22 )\end{matrix}$$

where k indexes the heads, aₖ is the gating value for head k, and xᵢ^{next} is the updated embedding generated by head k for input embedding xᵢ.

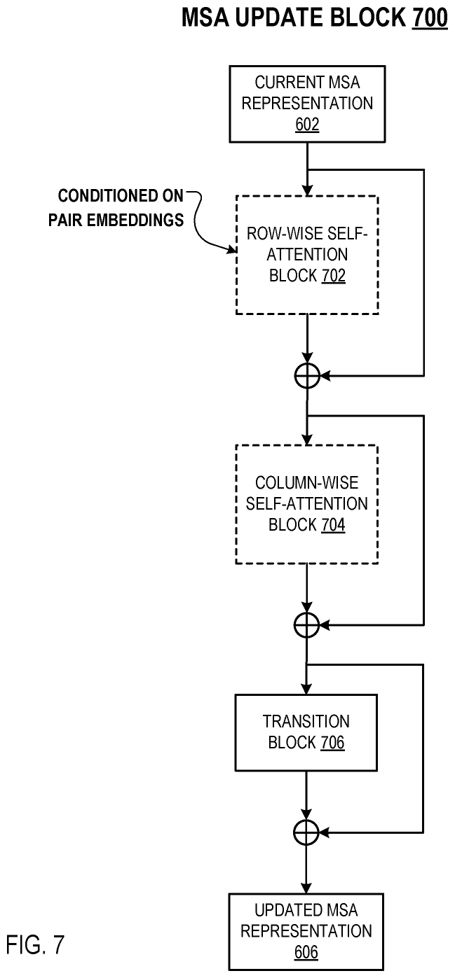

An example architecture of a MSA update block 700 that uses self-attention blocks conditioned on the pair embeddings is described with reference to FIG. 7. The example MSA update block described with reference to FIG. 7 updates the current MSA representation based on the current pair embeddings by processing the rows of the current MSA representation using a self-attention block that is conditioned on the current pair embeddings.

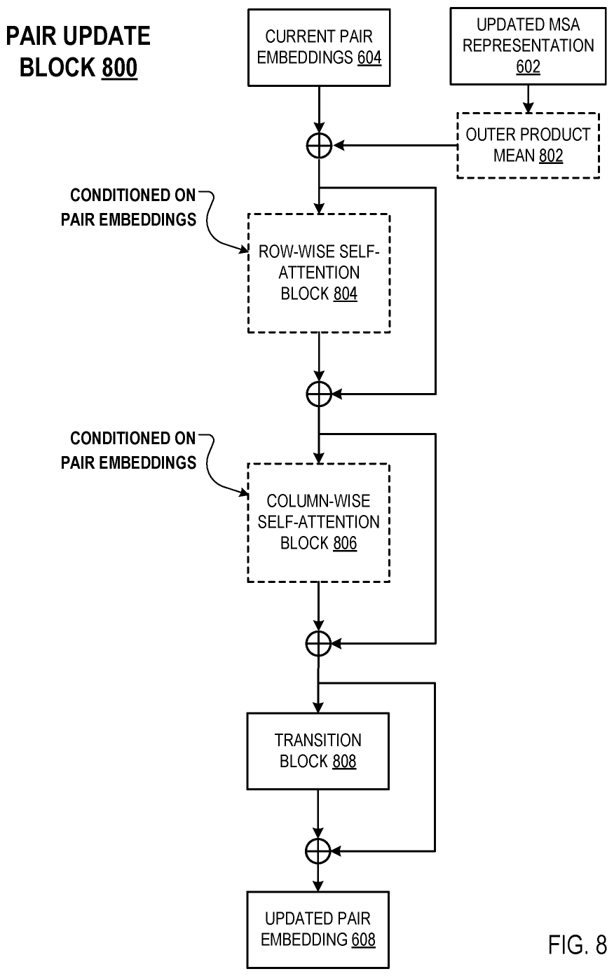

An example architecture of a pair update block 800 that uses self-attention blocks conditioned on the pair embeddings is described with reference to FIG. 8. The example pair update block described with reference to FIG. 8 updates the current pair embeddings based on the updated MSA representation by computing an outer product mean of the updated MSA representation, adding the result of the outer product mean to the current pair embeddings, and processing the current pair embeddings using self-attention blocks that are conditioned on the current pair embeddings.

FIG. 7 shows an example architecture of a MSA update block 700. The MSA update block 700 is configured to receive the current MSA representation 602, to update the current MSA representation 602 based (at least in part) on the current pair embedding.

To update the current MSA representation 602, the MSA update block 700 updates the embeddings in each row of the current MSA representation using a self-attention operation (i.e., a “row-wise” self-attention operation) that is conditioned on the current pair embeddings. More specifically, the MSA update block 700 provides the embeddings in each row of the current MSA representation 602 to a “row-wise” self-attention block 702 that is conditioned on the current pair embeddings, e.g., as described with reference to FIG. 6, to generate updated embeddings for each row of the current MSA representation 602. Optionally, the MSA update block can add the input to the row-wise self-attention block 702 to the output of the row-wise self-attention block 702. Conditioning the row-wise self-attention block 702 on the current pair embeddings enables the MSA update block 700 to enrich the current MSA representation 602 using information from the current pair embeddings.

The MSA update block then updates the embeddings in each column of the current MSA representation using a self-attention operation (i.e., a “column-wise” self-attention operation) that is not conditioned on the current pair embeddings. More specifically, the MSA update block 700 provides the embeddings in each column of the current MSA representation 602 to a “column-wise” self-attention block 704 that is not conditioned on the current pair embeddings to generate updated embeddings for each column of the current MSA representation 602. As a result of not being conditioned on the current pair embeddings, the column-wise self-attention block 704 generates updated embeddings for each column of the current MSA representation using attention weights (e.g., as described with reference to equations (18)-(19)) rather than biased attention weights (e.g., as described with reference to equation (21)). Optionally, the MSA update block can add the input to the column-wise self-attention block 704 to the output of the column-wise self-attention block 704.

The MSA update block then processes the current MSA representation 602 using a transition block, e.g., that applies one or more fully-connected neural network layers to the current MSA representation 602. Optionally, the MSA update block 700 can add the input to the transition block 706 to the output of the transition block 706.

The MSA update block can output the updated MSA representation 606 resulting from the operations performed by the row-wise self-attention block 702, the column-wise self-attention block 704, and the transition block 706.

FIG. 8 shows an example architecture of a pair update block 800. The pair update block 800 is configured to receive the current pair embeddings 604, and to update the current pair embeddings 604 based (at least in part) on the updated MSA representation 602.

To update the current pair embeddings 604, the pair update block 800 applies an outer product mean operation 802 to the updated MSA representation 602 and adds the result of the outer-product mean operation 802 to the current pair embeddings 604.

Generally, the updated MSA representation 602 encodes information about the correlations between the identities of the amino acids in different positions among a set of evolutionarily-related amino acid chains. The information encoded in the updated MSA representation 602 is relevant to predicting the structure of the protein, and by incorporating the information encoded in the updated MSA representation into the current pair embeddings (i.e., by way of the outer product mean 802), the pair update block 800 can enhance the information content of the current pair embeddings.

After updating the current pair embeddings 604 using the updated MSA representation (i.e., by way of the outer product mean 802), the pair update block 800 updates the current pair embeddings in each row of an arrangement of the current pair embeddings into an N_{AA}·N_{AA} array using a self-attention operation (i.e., a “row-wise” self-attention operation) that is conditioned on the current pair embeddings. More specifically, the pair update block 800 provides each row of current pair embeddings to a “row-wise” self-attention block 804 that is also conditioned on the current pair embeddings, e.g., as described with reference to FIG. 6, to generate updated pair embeddings for each row. Optionally, the pair update block can add the input to the row-wise self-attention block 804 to the output of the row-wise self-attention block 804.

The pair update block 800 then updates the current pair embeddings in each column of the N_{AA}·N_{AA} array of current pair embeddings using a self-attention operation (i.e., a “column-wise” self-attention operation) that is also conditioned on the current pair embeddings. More specifically, the pair update block 800 provides each column of current pair embeddings to a “column-wise” self-attention block 806 that is also conditioned on the current pair embeddings to generate updated pair embeddings for each column. Optionally, the pair update block can add the input to the column-wise self-attention block 806 to the output of the column-wise self-attention block 806.

The pair update block 800 then processes the current pair embeddings using a transition block, e.g., that applies one or more fully-connected neural network layers to the current pair embeddings. Optionally, the pair update block 800 can add the input to the transition block 808 to the output of the transition block 808.

The pair update block can output the updated pair embeddings 608 resulting from the operations performed by the row-wise self-attention block 804, the column-wise self-attention block 806, and the transition block 808.

FIG. 9 shows an example embedding expansion system 900, e.g., that can be included in the protein structure prediction system 300 described with reference to FIG. 3. The embedding expansion system 900 is an example of a system implemented as computer programs on one or more computers in one or more locations in which the systems, components, and techniques described below are implemented.

The system 900 is configured to process a set of pair embeddings 404 for a first amino acid chain in the protein to generate a set of interaction embeddings 210.

To generate the interaction embeddings 210, the system 900 generates a respective copy of the pair embeddings 404 for each amino acid chain in the protein. For example, the system 900 may generates copies 904-A-N of the pair embeddings, where each set of pair embeddings 904-A-N corresponds to a respective amino acid chain in the protein.

The system 900 generates respective “symmetry group relative position encoding” data 902 for each amino acid chain in the protein. The symmetry group relative position encoding data 902 defines a respective encoding vector for each amino acid chain of the protein, i.e., that distinguishes each amino acid chain from each other amino acid chain. For example, for a C4 symmetry group, the encoding vector for the first amino acid chain may be [1,0,0,0], the encoding vector for the second amino acid chain may be [0,1,0,0], the encoding vector for the third amino acid chain may be [0,0,1,0], and the encoder vector for the fourth amino acid chain may be [0,0,0,1].

For each amino acid chain in the protein, the system 900 processes the encoding vector defined by the symmetry group relative positon encoding 902 for the amino acid chain using an encoding neural network to generate a relative position embedding having the same number of channels as the pair embeddings. The system 900 can then add (or otherwise combine) the relative position embedding generated for the amino acid chain to each of the pair embeddings for the amino acid chain.

After combining the symmetry group relative position encoding data 902 with the pair embeddings 904-A-N for the amino acid chains, the system 900 update each set of pair embeddings 904-A-N by processing each set of pair embeddings 904-A-N using one or more pair update blocks 906-A-N. Each pair update block can update the pair embeddings using row-wise and column-wise self-attention blocks, e.g., as described with reference to FIG. 8.

After updating the pair embeddings 904-A-N using the pair update blocks 906-A-N, the system 900 can row-wise concatenate each set of pair embeddings 904-A-N to obtain the interaction embeddings 210. More specifically, each set of pair embeddings 904-A-N can be represented as an N_{AA}×N_{AA} array of pair embeddings, where N_{AA} is the number of amino acids in each amino acid chain, and the system 900 can concatenate these arrays of pair embeddings row-wise to obtain the N_{AA}×(N_{AA}·N_{C}) array of interaction embeddings, where N_{C} is the number of amino acid chains in the protein.

Optionally, the system 900 can update the array of interaction embeddings 210 by processing the array of interaction embeddings using one or more row-wise self-attention blocks and one or more column-wise self-attention blocks.

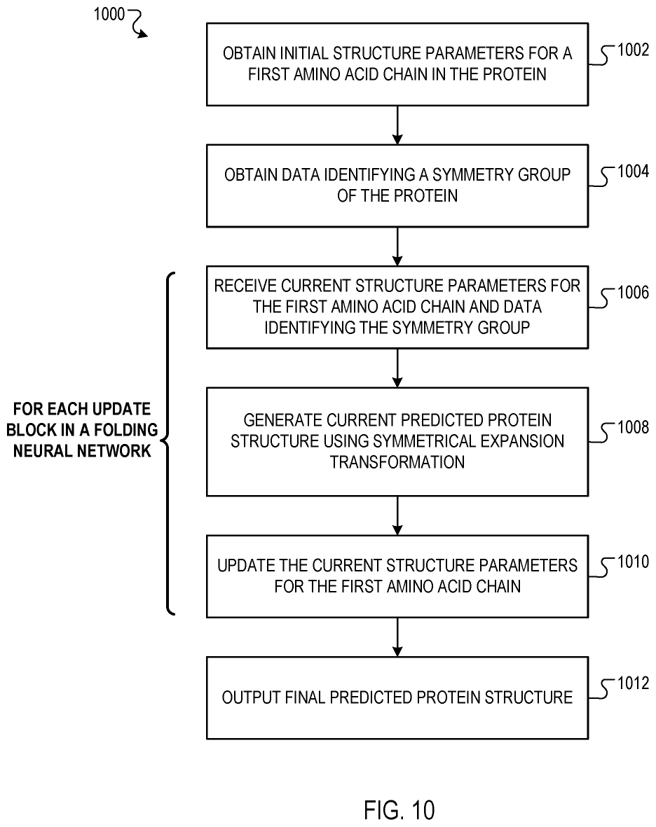

FIG. 10 is a flow diagram of an example process 1000 for predicting a structure of a protein that includes multiple amino acid chains. For convenience, the process 1000 will be described as being performed by a system of one or more computers located in one or more locations. For example, a protein structure prediction system, e.g., the protein structure prediction system 300 of FIG. 3, appropriately programmed in accordance with this specification, can perform the process 1000.

The system obtains initial structure parameters for a first amino acid chain in the protein (1002). Structure parameters for an amino acid chain in the protein define predicted three-dimensional spatial locations of amino acids in the amino acid chain in a structure of the protein.

The system obtains data identifying a symmetry group, where the protein is predicted to fold into a structure that is symmetrical with respect to the symmetry group (1004).

The system processes an input including the initial structure parameters for the first amino acid chain and the data identifying the symmetry group using a folding neural network

The folding neural network includes a sequence of update blocks. Steps 1006 — 1010, which are described next, are performed by each update block in the folding neural network.

The update block receives current structure parameters for the first amino acid chain and the data identifying the symmetry group (1006).

The update block applies a symmetrical expansion transformation to the current structure parameters for the first amino acid chain to generate respective current structure parameters for each other amino acid chain in the protein to define a current predicted structure of the protein that is symmetrical with respect to the symmetry group (1008).

The update block processes the current structure parameters for the amino acid chains in the protein, in accordance with values of the update block parameters of the update block, to update the current structure parameters for the first amino acid chain (1010).

The system uses the structure parameters for the first amino acid chain that are generated by the final update block to generate a final predicted structure of the protein that is symmetrical with respect to the symmetry group (1012).

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more data processing apparatus for predicting a structure of a protein that comprises a plurality of amino acid chains, the method comprising:
obtaining initial structure parameters for a first amino acid chain in the protein, wherein the structure parameters for the first amino acid chain in the protein define predicted three-dimensional (3D) spatial locations of amino acids in the first amino acid chain in a structure of the protein;
obtaining data identifying a symmetry group, wherein the protein is predicted to fold into a structure that is symmetrical with respect to the symmetry group;
processing an input comprising the initial structure parameters for the first amino acid chain and the data identifying the symmetry group using a folding neural network to generate an output that defines a final predicted structure of the protein that is symmetrical with respect to the symmetry group, wherein the folding neural network comprises a sequence of update blocks, wherein each update block in the sequence of update blocks has a plurality of update block parameters and performs operations comprising:
receiving current structure parameters for the first amino acid chain and the data identifying the symmetry group;
applying a symmetrical expansion transformation to the current structure parameters for the first amino acid chain to generate respective current structure parameters for each other amino acid chain in the protein to define a current predicted structure of the protein that is symmetrical with respect to the symmetry group; and
processing the current structure parameters for the amino acid chains in the protein, in accordance with values of the update block parameters of the update block, to update the current structure parameters for the first amino acid chain.

2. The method of claim 1, wherein the structure parameters for the first amino acid chain in the protein include respective amino acid structure parameters for each amino acid in the first amino acid chain, wherein the amino acid structure parameters for each amino acid define a 3D spatial location and orientation of the amino acid in a frame of reference of the first amino acid chain.

3. The method of claim 1, wherein the structure parameters for the first amino acid chain in the protein include global structure parameters that define a 3D spatial location and orientation of the first amino acid chain in a frame of reference of the protein.

4. The method of claim 3, wherein applying the symmetrical expansion transformation to the current structure parameters for the first amino acid chain to generate respective current structure parameters for each other amino acid chain in the protein comprises, for each other amino acid chain in the protein:
generating the global structure parameters for the other amino acid chain by applying a predefined transformation to the global structure parameters for the first amino acid chain, wherein the predefined transformation depends on: (i) a number of amino acid chains in the protein, and (ii) the symmetry group; and
determining amino acid structure parameters for the amino acids in the other amino acid chain that match the amino acid structure parameters for the amino acids in the first amino acid chain.

5. The method of claim 1, wherein the symmetry group is a cyclic symmetry group, a dihedral symmetry group, or a cubic symmetry group.

6. The method of claim 3, wherein the input processed by the folding neural network further comprises: (i) a respective initial amino acid embedding for each amino acid in the first amino acid chain, and (ii) an initial global embedding of the first amino acid chain.

7. The method of claim 6, wherein the operations performed by each update block further comprise receiving a respective current amino acid embedding for each amino acid in the first amino acid chain and a current global embedding of the first amino acid chain; and
wherein processing the current structure parameters for the amino acid chains in the protein to update the current structure parameters for the first amino acid chain comprises:
updating the current amino acid embeddings and the current global embedding for the first amino acid chain based on the current structure parameters for the amino acid chains in the protein; and
updating the current structure parameters for the first amino acid chain based on updated amino acid embeddings and the updated global embedding for the first amino acid chain.

8. The method of claim 7, wherein updating the current amino acid embeddings for the first amino acid chain based on the current structure parameters for the amino acid chains in the protein comprises:
determining, for each other amino acid chain in the protein, a respective current amino acid embedding for each amino acid in the other amino acid chain based on the current amino acid embedding of a corresponding amino acid in the first amino acid chain; and
updating the current amino acid embeddings and the current global embedding for the first amino acid chain using attention over the current amino acid embeddings for the amino acid chains, wherein the attention over the current amino acid embeddings for the amino acid chains is conditioned on the current structure parameters for the amino acid chains.

9. The method of claim 8, wherein updating the current global embedding for the first amino acid chain using attention over the current amino acid embeddings for the amino acid chains comprises:
determining, for each amino acid in each amino acid chain, a respective attention weight between the current global embedding for the first amino acid chain and the current amino acid embedding for the amino acid based at least in part on: (i) the global structure parameters for the first amino acid chain, and (ii) the amino acid structure parameters for the amino acid and the global structure parameters for the amino acid chain of the amino acid; and
updating the current global embedding for the first amino acid chain based on: (i) the attention weights, and (ii) the current amino acid embeddings for the amino acid chains.

10. The method of claim 9, wherein for each amino acid in each amino acid chain, determining the attention weight between the current global embedding for the first amino acid chain and the current amino acid embedding for the amino acid comprises:
generating a geometric query embedding corresponding to the current global embedding for the first amino acid chain, comprising:
processing the current global embedding for the first amino acid chain using one or more neural network layers to generate a 3D embedding;
rotating and translating the 3D embedding into a frame of reference of the protein using the global structure parameters for the first amino acid chain;
generating a geometric key embedding corresponding to the amino acid, comprising:
processing the current amino acid embedding of the amino acid using one or more neural network layers to generate a 3D embedding; and
rotating and translating the 3D embedding into the frame of reference of the protein using the amino acid structure parameters for the amino acid and the global structure parameters for the amino acid chain of the amino acid; and
determining the attention weight based on a spatial distance between: (i) the geometric query embedding corresponding to the current global embedding for the first amino acid chain, and (ii) the geometric key embedding corresponding to the amino acid.

11. The method of claim 7, wherein updating the current structure parameters for the first amino acid chain based on the updated amino acid embeddings and the updated global embedding for the first amino acid chain comprises:
for each amino acid in the first amino acid chain, updating the amino acid structure parameters for the amino acid based on the updated amino acid embedding for the amino acid; and
updating the global structure parameters for the first amino acid chain based on the updated global embedding for the first amino acid chain.

12. A system comprising:
one or more computers; and
one or more storage devices communicatively coupled to the one or more computers, wherein the one or more storage devices store instructions that, when executed by the one or more computers, cause the one or more computers to perform operations for predicting a structure of a protein that comprises a plurality of amino acid chains, the method comprising:
obtaining initial structure parameters for a first amino acid chain in the protein, wherein the structure parameters for the first amino acid chain in the protein define predicted three-dimensional (3D) spatial locations of amino acids in the first amino acid chain in a structure of the protein;
obtaining data identifying a symmetry group, wherein the protein is predicted to fold into a structure that is symmetrical with respect to the symmetry group;
processing an input comprising the initial structure parameters for the first amino acid chain and the data identifying the symmetry group using a folding neural network to generate an output that defines a final predicted structure of the protein that is symmetrical with respect to the symmetry group, wherein the folding neural network comprises a sequence of update blocks, wherein each update block in the sequence of update blocks has a plurality of update block parameters and performs operations comprising:
receiving current structure parameters for the first amino acid chain and the data identifying the symmetry group;
applying a symmetrical expansion transformation to the current structure parameters for the first amino acid chain to generate respective current structure parameters for each other amino acid chain in the protein to define a current predicted structure of the protein that is symmetrical with respect to the symmetry group; and
processing the current structure parameters for the amino acid chains in the protein, in accordance with values of the update block parameters of the update block, to update the current structure parameters for the first amino acid chain.

13. One or more non-transitory computer storage media storing instructions that when executed by one or more computers cause the one or more computers to perform operations for predicting a structure of a protein that comprises a plurality of amino acid chains, the method comprising:
obtaining initial structure parameters for a first amino acid chain in the protein, wherein the structure parameters for the first amino acid chain in the protein define predicted three-dimensional (3D) spatial locations of amino acids in the first amino acid chain in a structure of the protein;
obtaining data identifying a symmetry group, wherein the protein is predicted to fold into a structure that is symmetrical with respect to the symmetry group;
processing an input comprising the initial structure parameters for the first amino acid chain and the data identifying the symmetry group using a folding neural network to generate an output that defines a final predicted structure of the protein that is symmetrical with respect to the symmetry group, wherein the folding neural network comprises a sequence of update blocks, wherein each update block in the sequence of update blocks has a plurality of update block parameters and performs operations comprising:
receiving current structure parameters for the first amino acid chain and the data identifying the symmetry group;
applying a symmetrical expansion transformation to the current structure parameters for the first amino acid chain to generate respective current structure parameters for each other amino acid chain in the protein to define a current predicted structure of the protein that is symmetrical with respect to the symmetry group; and
processing the current structure parameters for the amino acid chains in the protein, in accordance with values of the update block parameters of the update block, to update the current structure parameters for the first amino acid chain.

14. (canceled)

15. (canceled)

16. (canceled)

17. (canceled)

18. (canceled)

19. (canceled)

20. (canceled)

21. (canceled)

22. (canceled)

23. (canceled)

24. The non-transitory computer storage media of claim 13, wherein the structure parameters for the first amino acid chain in the protein include respective amino acid structure parameters for each amino acid in the first amino acid chain, wherein the amino acid structure parameters for each amino acid define a 3D spatial location and orientation of the amino acid in a frame of reference of the first amino acid chain.

25. The non-transitory computer storage media of claim 13, wherein the structure parameters for the first amino acid chain in the protein include global structure parameters that define a 3D spatial location and orientation of the first amino acid chain in a frame of reference of the protein.

26. The non-transitory computer storage media of claim 25, wherein applying the symmetrical expansion transformation to the current structure parameters for the first amino acid chain to generate respective current structure parameters for each other amino acid chain in the protein comprises, for each other amino acid chain in the protein:
generating the global structure parameters for the other amino acid chain by applying a predefined transformation to the global structure parameters for the first amino acid chain, wherein the predefined transformation depends on: (i) a number of amino acid chains in the protein, and (ii) the symmetry group; and
determining amino acid structure parameters for the amino acids in the other amino acid chain that match the amino acid structure parameters for the amino acids in the first amino acid chain.

27. The non-transitory computer storage media of claim 13, wherein the symmetry group is a cyclic symmetry group, a dihedral symmetry group, or a cubic symmetry group.

28. The non-transitory computer storage media of claim 25, wherein the input processed by the folding neural network further comprises: (i) a respective initial amino acid embedding for each amino acid in the first amino acid chain, and (ii) an initial global embedding of the first amino acid chain.

29. The non-transitory computer storage media of claim 28, wherein the operations performed by each update block further comprise receiving a respective current amino acid embedding for each amino acid in the first amino acid chain and a current global embedding of the first amino acid chain; and
wherein processing the current structure parameters for the amino acid chains in the protein to update the current structure parameters for the first amino acid chain comprises:
updating the current amino acid embeddings and the current global embedding for the first amino acid chain based on the current structure parameters for the amino acid chains in the protein; and
updating the current structure parameters for the first amino acid chain based on updated amino acid embeddings and the updated global embedding for the first amino acid chain.

30. The non-transitory computer storage media of claim 29, wherein updating the current amino acid embeddings for the first amino acid chain based on the current structure parameters for the amino acid chains in the protein comprises:
determining, for each other amino acid chain in the protein, a respective current amino acid embedding for each amino acid in the other amino acid chain based on the current amino acid embedding of a corresponding amino acid in the first amino acid chain; and
updating the current amino acid embeddings and the current global embedding for the first amino acid chain using attention over the current amino acid embeddings for the amino acid chains, wherein the attention over the current amino acid embeddings for the amino acid chains is conditioned on the current structure parameters for the amino acid chains.
